(19) United States
(12) Patent Application Publication
(10) Pub. No.: US 2026/0265325 A1
(43) Pub. Date: Sep. 10, 2026

(54) INTERLEUKIN-33 VARIANTS, COMPOSITIONS AND USES THEREOF

(71) Applicants: Slate Bio, Inc., Charlottesville, VA (US); UNIVERSITY OF VIRGINIA PATENT FOUNDATION, Charlottesville, VA (US)

(72) Inventors: Michelle SOLTERO HIGGIN, Charlottesville, VA (US); Rahul SHARMA, Charlottesville, VA (US)

(21) Appl. No.: 19/676,080

(22) Filed: May 13, 2026

Related U.S. Application Data
(63) Continuation of application No. PCT/US2024/055912, filed on Nov. 14, 2024.
(60) Provisional application No. 63/599,205, filed on Nov. 15, 2023.

(51) Int. Cl. C07K 14/54; A61K 38/00; A61P 37/06; C07K 14/55

(52) U.S. Cl. CPC C07K 14/54; A61P 37/06; C07K 14/55; A61K 38/00; C07K 2319/00; C07K 2319/31

(57) ABSTRACT

The present invention provides, among other things, improved interleukin-33 (IL-33) variants engineered for increased manufacturability and activity, for example, mutated at one or more of N60, C116, C97, C121 or C148 relative to a truncated IL-33. The present invention also provides a multifunctional fusion protein comprising an IL-33 variant associated with an IL-2 polypeptide and optionally, a half-life extension moiety. The present invention further provides compositions and methods for proliferation and activity of ST2+ regulatory T cells, for prophylaxis and treatment of autoimmune disease.

### CROSS-REFERENCE TO RELATED APPLICATIONS

This Application is a Continuation Application of International Application No. PCT/US2024/055912, filed on Nov. 14, 2024, which claims priority to U.S. Provisional Application No. 63/599,205 filed on Nov. 15, 2023; the contents of which are incorporated herein by reference for all purposes.

### INCORPORATION BY REFERENCE OF SEQUENCE LISTING

The present application is being filed along with a Sequence Listing submitted electronically in XML format. The Sequence Listing file “SLB-007US1_SL.xml” is created on May 11, 2026, which is 91,735 bytes in size; the contents of which are incorporated herein for all purposes.

### BACKGROUND OF THE INVENTION

Interleukin-33 (IL-33) is a cytokine that stays bound to chromatin in the nucleus in a majority of cells. It is released upon tissue damage or stress, for example, necrosis, injury, or exposure to pathogens or allergens, and serves as an ‘alarmin’ to alert the immune system. IL-33 has dual roles as both a proinflammatory and anti-inflammatory molecule (Yi, Xue-Mei et al., 2022).

IL-33 interacts with the suppression of tumorigenicity 2, ST2 receptor (IL-1RL1) and IL-1 receptor accessory protein (IL-1RAcP), both of which are widely expressed, by innate immune cells, for example, T helper 2 (Th2) cells, mast cells, activated leukocytes like regulatory T (Treg) cells, group 2 innate lymphoid cells (ILC2s), CD8+ T cells and natural killer (NK) cells (Liew et al., 2016).

IL-33 induces Th2 cytokine production from these cells and has been implicated in the pathogenesis of Th2-related disease such as asthma, atopic dermatitis and anaphylaxis. Basophils and eosinophils, which are also targets of IL-33, release several proinflammatory cytokines such as IL-1β, IL-4, IL-5, IL-6, IL-8, IL-13 and granulocyte macrophage colon-stimulating factor (GMSF). IL-33 also directly activates iNKT and NK cells in cooperation with IL-12.

A subset of Treg cells, ST2+ Treg cells binds IL-33 and are enriched in inflammatory diseases. ST2+ cells are tissue targeting and more suppressive or reparative than systemic Treg cells. IL-33 signals through NFkB and MAPK pathways and independently stimulates Treg cells. Further, IL-33/ST2 signaling plays diverse roles in the homeostasis of the majority of tissues including central nervous system (CNS) and has been found to have protective as well as pathogenic roles in CNS diseases, including neurodegenerative diseases, cerebrovascular diseases, infection, trauma, and ischemic stroke.

Oxidation of IL-33 leads to formation of disulfide bridges and its inability to bind ST2 receptor. Oxidized forms of IL-33 cannot signal via NFkB, but has been shown to bind through RAGE/EGFR ST2 independent pathway, for example in epithelial mucosa, leading to inflammation, reduced tissue repair and chronic obstructive pulmonary disease (COPD) (Strickson et al., 2023).

On the other hand, IL-33 has shown various protective anti-inflammatory effects in cardiovascular diseases such as atherosclerosis, obesity, type 2 diabetes and cardiac remodeling. Further, an IL-33 IL-2 hybrid cytokine has been found to induce prolonged remission of mouse lupus nephritis (Stremska et al., 2019) and showed protective effects in murine acute graft vs host disease (aGVHD) (Venkatadri et al., 2021).

Owing to the breadth of target immune cells and the dual nature of IL-33 activity, there is a need for recombinant IL-33 with improved activity and stability that can be targeted to specific tissues for therapeutic use with minimal side-effects, in treating a variety of diseases, for example, inflammation, autoimmune disease and various types of cancers.

### SUMMARY OF THE INVENTION

The present invention provides, among other things, interleukin-33 variants that are engineered to have improved properties for therapeutic use, for example, having improved activity, stability and/or manufacturability relative to wild-type. Among other things, the human interleukin-33 (IL-33) variants disclosed herein have improved recombinant expression, reduced aggregation, leading to improved purification and yield, enhancing manufacturability. The IL-33 variants also show improved activity and potency and increased stability, maintaining potency after storage at various temperatures (e.g., 2-8° C., 25° C.).

In another aspect, also provided herein are multifunctional proteins comprising IL-33 variants of the present invention, associated together with a second polypeptide (e.g., as a fusion protein, and in some embodiments, via a linker) further comprising a half-life extension moiety that in combination, induce proliferation and/or activity of immune cells, for example, regulatory T cells or effector T cells, through additive or synergistic effects. In some embodiments, the second polypeptide is a cytokine (e.g., IL-2). In some embodiments, the second polypeptide is an antibody. In some embodiments, the second polypeptide is a therapeutic polypeptide.

In some embodiments, IL-33 variants described herein are associated with a second therapeutic polypeptide (e.g., a cytokine, antibody, scFv, Fc, nanobodies, peptide tags, chemokines, defensins, neuropeptides, bioactive peptides, vasoactive peptides, acute-phase proteins, toxins) or lipid mediators and polysaccharides among other things). In some embodiments, IL-33 variants are fused with an IL-2 polypeptide in a multifunctional fusion protein that expands or stimulates immune cells, e.g., regulatory T cells or effector T cells for various downstream effects. For example, in combination with IL-2, IL-33 variants induce proliferation and activity of regulatory Treg cells, which are mobilized to local tissues, resulting in a safe, potent and durable immune response for treating autoimmune disease. In other embodiments, IL-33 variants are fused with a second therapeutic polypeptide e.g., an IL-2 superkine variant, that stimulates proliferation and/or activity of T effector cells, for example, for use in treating cancer.

In some aspects, provided herein is a recombinant human interleukin-33 (IL-33) variant comprising at least one amino acid substitution at N60, C97, C116, C121, or C148, wherein the IL-33 has improved activity and/or manufacturability relative to wild-type IL-33.

In some embodiments, the recombinant human interleukin-33 (IL-33) variant comprises one, two, three, four or five amino acid substitutions at N60, C97, C116, C121 and C148, wherein the IL-33 has improved activity and/or manufacturability relative to wild-type IL-33.

In some aspects, provided herein is a recombinant human interleukin-33 (IL-33) variant comprising an amino acid substitution at N60 and any one of C97, C116, C121, or C148.

In some aspects, provided herein is a recombinant human interleukin-33 (IL-33) variant comprising an amino acid substitution C116F and an additional amino acid at any one of C97, C121, or C148.

In some aspects, provided herein is a recombinant human interleukin-33 (IL-33) variant comprising an amino acid substitution at position N60, wherein the substitution is selected from N60S, N60D, N60A, N60Q, N60T, or N60E.

In some embodiments, the recombinant human IL-33 variant comprises an amino acid substitution at position C97, wherein the substitution is selected from C97G, C97A or C97V.

In some embodiments, the recombinant human IL-33 variant comprises an amino acid substitution at position C116, wherein the substitution is selected from C116A, C116F or C116Y.

In some embodiments, the recombinant human IL-33 variant comprises an amino acid substitution at position C121, wherein the substitution is C121A or C121S. In one example, the substitution at position C121 is C121S.

In some embodiments, the recombinant human IL-33 variant comprises an amino acid substitution at position C148, wherein the substitution is selected from C148A, C148G, C148S, C148Y or C148N.

In some embodiments, the recombinant human IL-33 variant comprises the amino acid substitutions of C97G and C116F.

In some embodiments, the recombinant human IL-33 variant comprises the amino acid substitutions of C97G. C116F, and C148G.

In some embodiments, the recombinant human IL-33 variant comprises the amino acid substitutions of C97G, C116F, C121S and C148G.

In some embodiments, the recombinant human IL-33 variant comprises the amino acid substitutions of (a) N60D, C97G and C116F; (b) N60D, C97G. C116F, and C148G; or (c) N60D, C97G, C116F, C121S and C148G.

In some embodiments, the recombinant human IL-33 variant is a truncated IL-33.

In some embodiments, the recombinant human IL-33 variant polypeptide comprises the sequence of any one of SEQ ID NOs: 2-8, and SEQ ID NOs. 82-83.

In some embodiments, provided herein is a fusion protein comprising IL-33.

In some embodiments, the fusion protein further comprises IL-2.

In some embodiments, the fusion protein further comprises a half-life extension moiety.

In some aspects, provided herein is a multifunctional fusion protein comprising: an IL-2 polypeptide, an IL-33 polypeptide, and a half-life extension moiety, wherein the half-life extension moiety is located at the N-terminus or C-terminus of the fusion protein.

In some aspects, provided herein is a multifunctional fusion protein comprising: an IL-2 polypeptide, an IL-33 polypeptide, and a half-life extension moiety, wherein the half-life extension moiety is not an Fc domain of IgG.

In some aspects, provided herein is a multifunctional fusion protein, wherein the IL-33 polypeptide is a variant wherein the variant has improved activity and/or manufacturability relative to wild-type IL-33.

In some aspects, provided herein is a multifunctional fusion protein of any one of the preceding claims, comprising at least one amino acid substitution at N60, C97, C116, C121, or C148, and wherein the substitution at any one of the cysteines C97, C116, C121, or C148 is not a serine.

In some embodiments, the multifunctional fusion protein comprises an amino acid substitution at N60 and any one of C97, C116, C121, or C148.

In some embodiments, the multifunctional fusion protein comprises an amino acid substitution at C116F and an additional amino acid at any one of C97, C121, or C148.

In some embodiments, the multifunctional fusion protein comprises an amino acid substitution at position N60, wherein the substitution is selected from N60S, N60D, N60A, N60Q, N60T, or N60E.

In some embodiments, the multifunctional fusion protein comprises an amino acid substitution at position C97, wherein the substitution is selected from C97G, C97A or C97V.

In some embodiments, the multifunctional fusion protein comprises an amino acid substitution at position C116, wherein the substitution is selected from C116A, C116F or C116Y.

In some embodiments, the multifunctional fusion protein comprises an amino acid substitution at position C121, wherein the substitution is C121A or C121S. In one example, the substitution at position C121 is C121S.

In some embodiments, the multifunctional fusion protein comprises an amino acid substitution at position C148, wherein the substitution is selected from C148A, C148G, C148S, C148Y or C148N.

In some embodiments, the multifunctional fusion protein comprises the amino acid substitutions of C97G and C116F.

In some embodiments, the multifunctional fusion protein comprises an amino acid substitution of C97G, C116F, and C148G.

In some embodiments, the multifunctional fusion protein comprises an amino acid substitution of C97G, C116F, C121S and C148G.

In some embodiments, the multifunctional fusion protein comprises the amino acid substitutions of (a) N60D, C97G and C116F; (b) N60D, C97G. C116F, and C148G; or (c) N60D, C97G, C116F, C121S and C148G.

In some embodiments of the multifunctional fusion protein, the IL-33 is truncated.

In some embodiments of the multifunctional fusion protein, the IL-33 polypeptide comprises the sequence of any one of SEQ ID NOs: 2-8, and SEQ ID NOs. 81-82.

In some embodiments of the multifunctional fusion protein, the IL-2 polypeptide is a variant IL-2.

In some embodiments of the multifunctional fusion protein, the IL-2 polypeptide comprises N88R, N88S, C125S, V69A or Q74P.

In some embodiments of the multifunctional fusion protein, the half-life extension moiety is selected from the group consisting of monomethoxy poly-ethylene glycol (PEG), albumin, transferrin, Fc domain of IgG, highly sialylated peptide, XTEN, V-P-G-x-G (ELP), proline-alanine-serine (PAS) polymer, homo amino acid polymer (HAP) and gelatin-like protein (GLK).

In some embodiments, the PEG is linear chain or branched chain.

In some embodiments, the albumin is human serum albumin (I).

In some embodiments, the highly sialylated peptide is carboxy terminal peptide (CTP) consisting of the sequence of FQSSSS* KAPPPS*LPSPS*RLPGPS*DTPILPQ (SEQ ID NO: 51).

In some embodiments, the homo amino acid polymer consists of glycine.

In some embodiments, the Fc domain comprises 90% identity to SEQ ID NO: 54.

In some embodiments of the multifunctional fusion protein, wherein the Fc domain comprises 100% identity to the wild-type sequence of SEQ ID NO: 54.

In some embodiments of the multifunctional fusion protein, wherein the Fc domain comprises one or more mutations that reduce effector function.

In some embodiments of the multifunctional fusion protein, wherein the one or more mutations are selected from N297A, N297Q, N297G, L234A, L235E, G237A, P238S, H268Q, H268A, V309L, A330S and P331S.

In some embodiments of the multifunctional fusion protein, the IL-2 and IL-33 polypeptides are 10 to 100 angstrom units (Å) apart.

In some embodiments, the multifunctional protein further comprises a linker.

In some embodiments of the multifunctional fusion protein, the linker is a peptide linker or a chemical linker.

In some embodiments, the linker comprises a sequence of GGGGS (SEQ ID NO: 29) or GGGGSGGGGSGGGGS (SEQ ID NO: 30).

In some embodiments, the multifunctional fusion protein further comprises a tag at the N-terminus or C-terminus.

In some embodiments, the tag is a Flag tag.

In some embodiments, the multifunctional fusion protein comprises the sequence of any one of SEQ ID NO: 58-79.

In some embodiments, the fusion protein is lyophilized.

In some embodiments, provided herein is a polynucleotide encoding the IL-33 polypeptide or the multifunctional fusion protein described herein.

In some embodiments, provided herein is a gene therapy vector comprising the polynucleotide described herein.

In some embodiments, the gene therapy vector is a viral vector or a non-viral vector.

In some embodiments, the viral vector is an adeno-associated virus vector, an adenovirus vector or a lentivirus vector.

In some embodiments, provided herein is an engineered host cell comprising the polynucleotide or the vector described herein.

In some embodiments, the cell is a mammalian cell.

In some embodiments, the cell is a bacterial cell, insect cell or yeast cell.

In some embodiments, provided herein is a microparticle comprising the polynucleotide or the multifunctional fusion protein described herein.

In some embodiments, the microparticle is a liposome, extracellular vesicle or a lipid nanoparticle.

In some embodiments, provided herein is a pharmaceutical composition comprising the multifunctional fusion protein described herein.

In some embodiments, provided herein is a method of treating autoimmune disease, the method comprising administering to a subject in need thereof a therapeutically effective dose of the multifunctional fusion protein or the pharmaceutical composition or the engineered cell described herein.

In some embodiments, provided herein is a method of treating an autoimmune disease, the method comprising administering to a subject in need thereof a therapeutically effective dose of a multifunctional fusion of an IL-2 polypeptide, an IL-33 polypeptide, and a half-life extension moiety, wherein the half-life extension moiety is located at the N-terminus or C-terminus of the fusion protein.

In some embodiments, the administration is subcutaneous, intramuscular or intravenous.

In some embodiments, the administration is subcutaneous.

In some embodiments, the administration stimulates proliferation and/or activation of immune cells.

In some embodiments, the immune cells are ST2+ cells, Foxp3-ST2+, Foxp3-ST2-Tr1 cells, Th3 cells, ILC2, macrophages, MDSCs, regulatory B cells, B1B cells, or tolerogenic dendritic cells.

In some embodiments, the ST2+ cells are ST2+ T regulatory cells, tissue resident ST2+ cells or a combination of tissue resident ST2+ and CD25^{bright} cells.

In some embodiments, the proliferation and/or activation of ST2+ T regulatory cells is at least greater than 100-fold relative to a control.

In some embodiments, the administration leads to coexpression of IL-2 and IL-33 in the same tissue.

In some embodiments, IL-2 and IL-33 synergistically show increased potency and/or increased avidity relative to IL-2 or IL-33 alone.

In some embodiments, administration increases local immune tolerance in a tissue microenvironment.

Any aspect or embodiment described herein can be combined with any other aspect or embodiment as disclosed herein. While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

Other features and advantages of the invention will be apparent from the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The following figures are for illustration purposes only and not for limitation.

FIG. 1A is an SDS-PAGE gel showing expression of an exemplary an IL-33 variant in reducing and non-reducing conditions of about 93% purity. FIG. 1B is a chromatogram showing a peak corresponding to 99.9% pure multifunctional protein, comprising an IL-33 variant.

FIG. 2A is a graph showing IL-33 activity of IL-33 WT, IL-33 variant and an exemplary multifunctional protein, SLT8, as measured by NF-κB/AP-1-inducible secreted embryonic alkaline phosphatase (SEAP) reporter gene levels (indicative of IL-33 signaling) in an HEK-Blue™ IL-33 reporter cell line (InvivoGen). FIG. 2B is a graph showing IL-2 activity of IL-2 WT, IL-33 variant and an exemplary multifunctional protein, SLT8, as measured by STAT5-inducible secreted embryonic alkaline phosphatase (SEAP) reporter gene levels (indicative of IL-2 signaling) in an HEK-Blue™ IL-33 reporter cell line (InvivoGen).

FIG. 3A is a bar graph showing the number of ST2+ Treg cells at day 10, measured from mice subcutaneously injected with a dose of 10 ug/kg or 100 ug/kg of exemplary bifunctional protein, SLT8 (also referred to as SLT-008/SLT008) or SLT27 (also referred to as SLT-027/SLT027) at days 0, 1 or 2. Both SLT8 and SLT27 showed a dose-dependent expansion of ST2+ Treg cells by about 5-10 fold. FIG. 3B is a bar graph showing the number of total Treg cells at day 10, measured from mice subcutaneously injected with a dose of 10 ug/kg or 100 ug/kg of exemplary bifunctional protein, SLT8 or SLT27 at days 0, 1 or 2. Both SLT8 and SLT27 showed a dose-dependent expansion of total Treg cells by about 2-3 fold.

FIG. 4A is a bar graph of the number of eosinophils measured at days 4, 6 and 10 from mice subcutaneously injected with a dose of 10 μg/kg or 100 μg/kg of exemplary bifunctional protein, SLT8 or SLT27 at days 0, 1 or 2. SLT8 and SLT27 showed comparable amounts of eosinophils at the timepoints tested. FIG. 4B is a bar graph of the percentage of T effector cells measured at days 4, 6 and 10 from mice subcutaneously injected with a dose of 10 ug/kg or 100 ug/kg of exemplary bifunctional protein, SLT8 or SLT27 at days 0, 1 or 2. SLT8 and SLT27 showed comparable amounts of T effector cells at the timepoints tested.

### DEFINITIONS

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The terms “e.g.,” and “i.e.,” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

Unless specifically stated or evident from context, as used herein, the term “about” is understood as within a range of normal tolerance in the art, for example within 2 standard deviations of the mean. “About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein reflects normal fluctuations that can be appreciated by a skilled artisan.

Fusion protein, as used herein, generally refers to a fusion polypeptide molecule comprising an IL-33 molecule and an IL-2 molecule, wherein the components of the fusion protein are linked to each other by peptide-bonds, either directly or through peptide linkers. In some embodiments, the fusion protein also comprises a half-life extension moiety (e.g., an Fc region, PEG, etc.) and a linker.

Fused refers to components that are linked by peptide bonds, either directly or via one or more peptide linkers.

Affinity or binding affinity refers to the strength of the sum total of non-covalent interactions between a single binding site of a molecule and its binding partner. Unless indicated otherwise, as used herein, binding affinity refers to intrinsic binding affinity which reflects a 1:1 interaction between members of a binding pair. The affinity of a molecule X for its partner Y can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR). Specific binding means that the binding is selective for the protein and can be discriminated from unwanted or non-specific interactions. The ability of a protein to bind to a specific binding partner can be measured either through an enzyme-linked immunosorbent assay (ELISA) or other techniques familiar to one of skill in the art, e.g., Surface Plasmon Resonance (SPR) technique (analyzed on a BIAcore instrument), and traditional binding assays. In some embodiments, the protein has a dissociation constant (D) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). Reduced binding, for example reduced binding of a cytokine (e.g., IL-2 or IL-33) to an IL-2 or an IL-33 receptor, refers to a decrease in affinity for the respective interaction, as measured for example by SPR. For clarity the term includes also reduction of the affinity to zero (or below the detection limit of the analytic method), i.e., complete abolishment of the interaction. Conversely, increased binding refers to an increase in binding affinity for the respective interaction.

Autoimmune disease refers to a non-malignant disease or disorder arising from and directed against an individuals' own tissues. Examples of autoimmune diseases or disorders include, but are not limited to, inflammatory responses such as inflammatory skin diseases including psoriasis and dermatitis (e.g., atopic dermatitis); responses associated with inflammatory bowel disease (such as Crohn's disease and ulcerative colitis); dermatitis; allergic conditions such as eczema and asthma; rheumatoid arthritis; systemic lupus erythematosus (SLE) (including but not limited to lupus nephritis, cutaneous lupus); diabetes mellitus (e.g., type 1 diabetes mellitus or insulin dependent diabetes mellitus); multiple sclerosis and juvenile onset diabetes. Additional examples of autoimmune diseases include, for example, multiple sclerosis (MS), lupus, ankylosing spondylitis, arthritis, colitis, type 1 diabetes, Crohn's disease, heart disease, graft versus host disease, complications from immune response in pregnancy, allergies, rejection of cell or solid organ transplant, Amyotrophic lateral sclerosis (ALS), and myasthenia gravis.

Interleukin-2 or IL-2 as used herein, refers to any native IL-2 from any vertebrate source, including mammals such as primates (e.g., humans) and rodents (e.g., mice and rats), unless otherwise indicated. The term encompasses unprocessed IL-2 as well as any form of IL-2 that results from processing in the cell. The term also encompasses naturally occurring variants of IL-2, e.g., splice variants or allelic variants. The amino acid sequence of an exemplary human IL-2 is shown in SEQ ID NO: 9. Unprocessed human IL-2 additionally comprises an N-terminal 20 amino acid signal peptide (SEQ ID NO: 10), which is absent in the mature IL-2 molecule. Wild-type IL-2 or native IL-2, also termed wild-type IL-2, refers to a naturally occurring IL-2. The sequence of a native human IL-2 molecule is shown in SEQ ID NO: 9.

Interleukin-2 or IL-2 variant as used herein, refers to an IL-2 mutein that comprises one or more amino acid mutations relative to wild-type. In some embodiments, the one or more mutations do not alter IL-2 receptor binding compared to the naturally occurring, native IL-2, such as e.g., a substitution of cysteine at a position corresponding to residue 125 of human IL-2 to alanine. In some embodiments wild-type IL-2 for the purpose of the present invention comprises one or more amino acid substitutions selected from T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, 192R, E95G, C125A, C125S Q1261, Q126L, and Q126F. In some embodiments of the present invention, the IL-2 polypeptide comprises one or more amino acid substitutions selected from V69A, Q47P, N88D, N88R and C125S. In some embodiments, the IL-2 polypeptide comprises amino acid substitutions of N88D and C125S.

CD25 or IL-2 receptor α as used herein, refers to any native CD25 from any vertebrate source, including mammals such as primates (e.g., humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g., splice variants or allelic variants. In some embodiments CD25 is human CD25.

High-affinity IL-2 receptor as used herein refers to the heterotrimeric form of the IL-2 receptor, consisting of the receptor γ-subunit (also known as common cytokine receptor γ-subunit, yc, or CD132), the receptor β-subunit (also known as CD122 or p70) and the receptor α-subunit (also known as CD25 or p55).

Intermediate-affinity IL-2 receptor or IL-2 receptor By refers to the IL-2 receptor including only the γ-subunit and the β-subunit, without the α-subunit (Olejniczak and Kasprzak, Med Sci Monit 14, RA179-189 (2008)).

Regulatory T cell or Treg cell refers to a specialized type of CD4+ T cell that can suppress the responses of other T cells (effector T cells). Treg cells are characterized by expression of CD4, the α-subunit of the IL-2 receptor (CD25), and the transcription factor forkhead box P3 (FOXP3) (Sakaguchi, Annu Rev Immunol 22, 531-62 (2004)) and play a critical role in the induction and maintenance of peripheral self-tolerance to antigens, including those expressed by tumors.

CD4+ T cells means CD4+ T cells other than regulatory T cells. Conventional CD4+ memory T cells are characterized by expression of CD4, CD3, but not FOXP3. Conventional CD4+ memory T cells are a subset of conventional CD4+ T cells, further characterized by lack of expression of CD45RA, in contrast to conventional CD4+ naïve T cells which do express CD45RA.

Selective activation of Treg cells is meant activation of Treg cells essentially without concomitant activation of other T cell subsets (such as CD4+T helper cells, CD8+ cytotoxic T cells, NK T cells) or natural killer (NK) cells. Activation may include induction of IL-2 receptor signaling (as measured e.g., by detection of phosphorylated STAT5a), induction of proliferation (as measured e.g., by detection of Ki-67) and/or up-regulation of expression of activation markers (such as e.g., CD25).

Peptide linker refers to a peptide comprising one or more amino acids, typically about 2-20 amino acids. Peptide linkers are known in the art or are described herein, as non-limiting examples, at Table 4.

Potency refers to the amount of a therapeutic agent needed to produce a given effect. Efficacy is the maximal effect that a drug produces irrespective of concentration (dose). Potency refers to the rate of drug-receptor binding and dissociation whereas efficacy refers to the resultant biological response. Potency is calculated by median effective concentration/dose KD, or EC50, or ED50. It refers to the concentration of therapeutic agent needed to activate 50% of the available receptors.

Modification refers to any manipulation of the peptide backbone (e.g., amino acid sequence) or the post-translational modifications (e.g., glycosylation) of a polypeptide.

Amino acid substitution refers to the replacement in a polypeptide of one amino acid with another amino acid. In one embodiment, an amino acid is replaced with another amino acid having similar structural and/or chemical properties, e.g., conservative amino acid replacements.

Conservative amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Non-conservative amino acid substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic). Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid substitution. A position N60 be indicated as N60S, 60S, S60, or Asn60Ser.

Percent (%) amino acid sequence identity with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that ‘program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Polynucleotide or nucleic acid as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label.

Percent identity of nucleic acid sequence: By a nucleic acid or polynucleotide having a nucleotide sequence at least, for example, 95% “identity” to a reference nucleotide sequence of the present invention, it is intended that the nucleotide sequence of the polynucleotide is identical to the reference sequence except that the polynucleotide sequence may include up to five-point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention is determined conventionally using known computer programs, such as the ones discussed above for polypeptides (e.g., ALIGN-2).

Variant as used herein, refers to a mutein comprising one or more amino acid substitutions relative to wild-type sequence. Mutations maybe any mutation described in the present application, or equivalents thereof. Interleukin-2 or IL-2 variant as used herein, refers to an IL-2 mutein that comprises one or more amino acid mutations relative to wild-type. Interleukin-33 or IL-33 variant as used herein, refers to an IL-33 mutein that comprises one or more amino acid mutations relative to wild-type.

Vector as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

Host cell, host cell line, and host cell culture are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. A host cell is any type of cellular system that can be used to generate the fusion proteins of the present invention. Host cells include cultured cells, e.g., mammalian cultured cells, such as CHO cells, BH cells, NS0 cells, SP2/0 cells, YO myeloma cells, P3X63 mouse myeloma cells, PER cells, PER. C6 cells or hybridoma cells, bacterial cells yeast cells, insect cells, and plant cells, to name only a few, but also cells comprised within a transgenic animal, transgenic plant or cultured plant or animal tissue.

Effective amount of an agent refers to the amount that is necessary to result in a physiological change in the cell or tissue to which it is administered.

Therapeutically effective amount of an agent, e.g., a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

Individual or subject is a mammal (e.g., human) administered an effective dose of the multifunctional protein.

Manufacturability as used herein, in the context of recombinant proteins (e.g., IL-2, IL-33, including variants, and IL233 fusion proteins), refers to biological activity, oxidation, solubility, stability and/or yield for making “druggable” recombinant proteins for therapeutic use.

Stability: As used herein, the term “stable” refers to the ability of the therapeutic agent (e.g., recombinant IL-2, IL-33, and IL233 fusion proteins) to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). The therapeutic agent essentially retains its physical and/or chemical integrity and biological activity upon storage and during processes subjected to the composition or formulation (such as freeze/thaw, mechanical mixing and lyophilization). For example, protein stability can be measured by formation of high molecular weight (HMW) aggregates, loss of enzyme activity, generation of peptide fragments and shift of charge profiles.

Pharmaceutical composition refers to a preparation which is in such form as to permit the biological activity of an active ingredient contained therein to be effective, and which contains no additional components which are unacceptably toxic to a subject to which the formulation would be administered. As used herein the term “pharmaceutical composition” refers to compositions comprising at least one active ingredient and optionally one or more pharmaceutically acceptable excipients.

Pharmaceutically acceptable carrier refers to an ingredient in a pharmaceutical composition, other than an active ingredient, which is nontoxic to a subject. A pharmaceutically acceptable carrier includes, but is not limited to, a buffer, excipient, stabilizer, or preservative.

Regulatory T cells also referred to as T Regulatory cells or Treg cells are a specialized subpopulation of T cells that act to suppress immune response, thereby maintaining homeostasis and self-tolerance. The main subsets of CD4+ regulatory T (Tr) cells are the CD4+CD25+Tr cells and the type 1 regulatory (Tr1) cells. Tregs inhibit T cell proliferation and cytokine production and play a critical role in preventing autoimmunity.

ST2+ Treg cells are a subset of Treg cells that are also referred to as “reparative” or “suppressive” or “tissue resident” Treg cells. ST2+ Tregs contain the ST2 cell surface marker, which is a component of a cytokine receptor also known interleukin 1 receptor-like 1 protein (IL1RL1), which is a subunit of the IL-33 receptor.

Signal peptide: As used herein, a “signal sequence” or “signal peptide” is a polynucleotide or polypeptide, respectively, which is from about 9 to 200 nucleotides (about 3-66 amino acids) in length, that is incorporated at the 5′ terminus of the coding region or the N-terminus polypeptide encoded, respectively. In some embodiments, addition of these sequences result in trafficking of the encoded polypeptide to the endoplasmic reticulum through one or more secretory pathways. Some signal peptides are cleaved from the protein by signal peptidase after the proteins are transported.

Substantially refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term substantially is therefore used herein to capture the potential lack of completeness inherent in many biological and chemical phenomena.

Synergistic Effect: As used herein, the term “synergistic effect” refers to an overall effect of a biological moiety, such as, for example, a fusion protein, that is greater than the sum of individual effects of the components, such as, the peptides included in the fusion protein. For the present invention, an example of synergistic effect is a greater effect of a multifunctional protein comprising an IL-33 variant and IL-2 than either cytokine by itself.

Treatment (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishing of any direct or indirect pathological consequences of the disease, preventing autoimmune disease, decreasing the rate of disease progression, amelioration or palliation of the disease state, and remission or improved prognosis. In some embodiments, antibodies of the invention are used to delay development of a disease or to slow the progression of a disease.

Unless otherwise defined, all technical and scientific terms used herein have the same meaning as commonly understood by one of ordinary skill in the art to which this application belongs and as commonly used in the art to which this application belongs; such art is incorporated by reference in its entirety. In the case of conflict, the present Specification, including definitions, will control.

### DETAILED DESCRIPTION OF THE INVENTION

The present invention provides, among other things, IL-33 variants and multifunctional proteins comprising said IL-33 variants associated with a second polypeptide (e.g., a second therapeutic agent, e.g., a cytokine, e.g., IL-2) that are engineered to have improved properties such as improved activity and/or manufacturability relative to wild-type IL-33. Among other things, the IL-33 variants of the present invention are easily expressed recombinantly, show reduced aggregation, leading to improved purification and yield, thereby enhancing manufacturability. The variants described herein also showed beneficial properties such as increased stability, increased activity and increased potency.

The IL-33 variants of the present invention were designed and constructed by site-specific mutagenesis, e.g., by selectively replacing cysteine residues with other residues that are not capable of forming disulfide linkages or by mutating potential glycosylation sites. Rather than use a shot-gun approach that replaced all cysteine residues generating an IL-33 that showed profoundly increased half-life and persistence in serum, the inventors of the present invention carefully generated IL-33 variants by discerning mutagenesis that balanced functional properties such as persistence in serum and optimal activity with species conservation, safety parameters for in vivo administration of a recombinant IL-33 protein, for example, at one or more of C97, C116, C121, or C148. Truncated IL-33 domains comprising a point mutation, for example, at C116 are less likely to form disulfide linkages with other key cysteine residues in the truncated IL-33 domain and/or with cysteine residues in the IL-2 domain and/or a half-life extension moiety (e.g., Fc domain). In some embodiments, the IL-33 variants of the present invention show reduced oxidation and improved activity relative to IL-33 WT.

Further, truncated IL-33 proteins comprising a point mutation at an asparagine residue, N60, for example are less susceptible to glycosylation, and show improved features including reduced aggregation, slower clearance in vivo, and higher activity relative to a wild-type truncated IL-33. In some embodiments, the inventors of the present invention have combined mutations of the glycosylation site with selective mutation of cysteines to generate IL-33 variants with improved properties such as manufacturability and/or activity.

The present invention also provides, among other things, multifunctional proteins comprising human interleukin-3 (IL-33) variants and IL-2 that are associated together (e.g., as a fusion protein, and in some embodiments, via a linker) further comprising a half-life extension moiety. Disclosed herein are the unexpected results of a synergistic effect of the combination of IL-2 and IL-33 in a single multifunctional protein, providing compositions and methods for selective proliferation of tissue-resident suppressive regulatory T cells, which are also referred to as reparative Treg cells or ST2+ Treg cells. In addition, CD25 bright and ST2+ CD25 bright double positive Treg cells are also expanded and/or activated. The present invention provides, among other things, compositions and methods for prophylaxis and treatment of autoimmune disease and inflammatory disorders.

Various aspects of the invention are described in detail in the following sections. The use of sections is not meant to limit the invention. Each section can apply to any aspect of the invention. In this application, the use of “or” means “and/or” unless stated otherwise.

### Interleukin-33 (IL-33) Variants

IL-33 is a pleiotropic cytokine, also referred to as “alarmin”, which is released upon tissue injury. Tissue-derived immune cells, including Tregs, innate lymphoid cells (ILC2s), mast cells constitutively express the IL-33 receptor, also known as the ST2 receptor. IL-33 also activates additional immune cells, such as Th2 cells, basophils, eosinophils, M2 macrophages, and dendritic cells. In addition, immune cells including NK cells, iNKT cells and neutrophils, constitutively express ST2. ST2 expression is inducible and regulated by the tissue microenvironment.

Human IL-33 is a 270 amino acid protein belonging to the IL-1 cytokine family, and is composed of two evolutionary conserved domains, the N-terminal nuclear domain and the C-terminal IL-1-like cytokine domain, separated by a divergent ‘protease sensor’ domain. The N-terminal domain (aa 1-65) is required for nuclear localization and comprises a chromatin-binding motif (CBM). The ‘protease sensor’ domain of IL-33 (aa 66 to 111) is a protease cleavage and activation site.

The C-terminal domain (cytokine domain, aa 112-270) possesses cytokine activity and has a three-dimensional structure similar to interleukin 1 (IL-1). The C-terminal is responsible for binding to the ST2 receptor along with a co-receptor IL-1 receptor accessory protein (IL-1RAcP) that facilitates the interaction. The IL-33-ST2 interaction is mediated by surface charge complementarity. Protease cleavage of IL-33 yields an 18 kDa C-terminal fragment, which is referred to herein as truncated IL-33. Truncated IL-33 by itself can stimulate Treg cells, ILCs, Th2 cells, macrophages, and dendritic cells, and thereby upregulate the expression of IL-2, IL-4, IL-5, and IL-13, and also reduce pro-inflammatory Th1 and Th17 responses. Fusion proteins comprising truncated WT IL-33 domains have been described in the art, for example, fusions with IL-2, U.S. Pat. No. 9,840,545B2; U.S. Pat. No. 10,851,145B2; and US20210261640A1; the disclosures of which are hereby incorporated by reference.

IL-33 Variants Having Improved Manufacturability and/or Activity

The present invention provides, among other things, IL-33 variants or IL-33 muteins having improved properties including manufacturability and/or activity.

In some aspects, provided herein is a recombinant human interleukin-33 (IL-33) variant comprising at least one amino acid substitution at N60, C97, C116, C121, or C148, wherein the substitution at C97, C116, or C148 is not a serine and wherein the IL-33 has improved activity and/or manufacturability relative to wild-type IL-33. As used herein, wild-type IL-33 used for comparison is a truncated IL-33 lacking site-specific mutations and the amino acid positions noted here are relative to truncated IL-33. In some embodiments, provided herein is a recombinant human interleukin-33 (IL-33) variant comprising an amino acid substitution at C97, wherein the substitution is not a serine and wherein the IL-33 has improved activity and/or manufacturability relative to wild-type IL-33. In some embodiments, provided herein is a recombinant human interleukin-33 (IL-33) variant comprising an amino acid substitution at C116, wherein the substitution is not a serine and wherein the IL-33 has improved activity and/or manufacturability relative to wild-type IL-33. In some embodiments, provided herein is a recombinant human interleukin-33 (IL-33) variant comprising an amino acid substitution at C121, wherein the IL-33 has improved activity and/or manufacturability relative to wild-type IL-33. In some embodiments, provided herein is a recombinant human interleukin-33 (IL-33) variant comprising an amino acid substitution at C148, wherein the substitution is not a serine and wherein the IL-33 has improved activity and/or manufacturability relative to wild-type IL-33. In some embodiments, provided herein is a recombinant human interleukin-33 (IL-33) variant comprising an amino acid substitution at N60, and wherein the IL-33 has improved activity and/or manufacturability relative to wild-type IL-33.

In some aspects, provided herein is a recombinant human interleukin-33 (IL-33) variant comprising an amino acid substitution at N60 and any one of C97, C116, C121, or C148. In some embodiments, provided herein is a recombinant human interleukin-33 (IL-33) variant comprising an amino acid substitution at N60 and C97. In some embodiments, provided herein is a recombinant human interleukin-33 (IL-33) variant comprising an amino acid substitution at N60 and C116. In some embodiments, provided herein is a recombinant human interleukin-33 (IL-33) variant comprising an amino acid substitution at N60 and C121. In some embodiments, provided herein is a recombinant human interleukin-33 (IL-33) variant comprising an amino acid substitution at N60 and C148.

In some aspects, provided herein is a recombinant human interleukin-33 (IL-33) variant comprising an amino acid substitution C116F and an additional amino acid substitution at any one of C97, C121, or C148. In some embodiments, provided herein is a recombinant human interleukin-33 (IL-33) variant comprising an amino acid substitution C116F and an additional amino acid substitution at C97. In some embodiments, provided herein is a recombinant human interleukin-33 (IL-33) variant comprising an amino acid substitution C116F and an additional amino acid substitution at C121. In some embodiments, provided herein is a recombinant human interleukin-33 (IL-33) variant comprising an amino acid substitution C116F and an additional amino acid substitution at C148.

In some aspects, provided herein is a recombinant human interleukin-33 (IL-33) variant comprising an amino acid substitution selected from N60S, N60D, N60A, N60T, N60Q, N60E, C97G, C97A, C97V, C116A, C116F, C116Y, C121A, C121S, C148A, C148S, C148Y, C148N and C148G. In some embodiments, provided herein is a recombinant human interleukin-33 (IL-33) variant comprising an amino acid substitution at position N60, wherein the substitution is N60S, N60D, N60A, N60Q, N60T or N60E. In some embodiments, provided herein is a recombinant human interleukin-33 (IL-33) variant comprising an N60D amino acid substitution.

In some embodiments, provided herein is a recombinant human interleukin-33 (IL-33) variant comprising an amino acid substitution at position C97, wherein the substitution is C97G, C97A or C97V. In some embodiments, provided herein is a recombinant human interleukin-33 (IL-33) variant comprising an C97G amino acid substitution.

In some embodiments, provided herein is a recombinant human interleukin-33 (IL-33) variant comprising an amino acid substitution at position C119, wherein the substitution is C116F or C116Y. In some embodiments, provided herein is a recombinant human interleukin-33 (IL-33) variant comprising an C116F amino acid substitution.

In some embodiments, provided herein is a recombinant human interleukin-33 (IL-33) variant comprising an amino acid substitution at position C121, wherein the substitution is C121S. In some embodiments, provided herein is a recombinant human interleukin-33 (IL-33) variant comprising an C148 amino acid substitution, wherein the substitution is C148G, C148S, C148Y or C148N. In some embodiments, provided herein is a recombinant human interleukin-33 (IL-33) variant comprising an C148G amino acid substitution.

In some embodiments, provided herein is a recombinant human interleukin-33 (IL-33) variant comprising an N60S and a C97G amino acid substitution. In some embodiments, provided herein is a recombinant human interleukin-33 (IL-33) variant comprising an N60D and a C97G amino acid substitution. In some embodiments, provided herein is a recombinant human interleukin-33 (IL-33) variant comprising an N60S and a C116F amino acid substitution. In some embodiments, provided herein is a recombinant human interleukin-33 (IL-33) variant comprising an N60D and a C116F amino acid substitution. In some embodiments, provided herein is a recombinant human interleukin-33 (IL-33) variant comprising an N60S, C97G and a C116F amino acid substitution. In some embodiments, provided herein is a recombinant human interleukin-33 (IL-33) variant comprising an N60D, C97G and a C116F amino acid substitution.

In some embodiments, the recombinant human IL-33 variant comprises the amino acid substitutions of C97G and C116F.

In some embodiments, the recombinant human IL-33 variant comprises an amino acid substitution of C97G, C116F, and C148G.

In some embodiments, the recombinant human IL-33 variant comprises an amino acid substitution of C97G, C116F, C121S and C148G.

In some embodiments, the recombinant human IL-33 variant is a truncated IL-33.

In some embodiments, the recombinant human IL-33 variant polypeptide comprises the sequence of any one of SEQ ID NOs: 2-8, and SEQ ID NOs: 82-83.

IL-33 is an immunomodulatory compound that on the one hand is a proinflammatory cytokine associated with acute inflammatory responses, also referred to as alarmin, and on the other hand via expression on Tregs, macrophages, ILC2, regulatory B cells, B1B cells and MDSC is anti-inflammatory and pro-resolving. In some embodiments, IL-33 variants described herein are associated with a second therapeutic agent. In some embodiments, the second therapeutic agent is selected from the group consisting of antibodies, scFv, Fc, nanobodies, peptide-tags, cytokines, chemokines, defensins, neuropeptides, bioactive peptides, vasoactive peptides, acute-phase proteins, toxins), lipid mediators and polysaccharides. In some embodiments, IL-33 variants are associated with an IL-2 polypeptide. In some embodiments, IL-33 variants are fused with an IL-2 polypeptide in a multifunctional fusion protein.

### Multifunctional Proteins Comprising IL-33 Variants

In some aspects, the present invention provides multifunctional proteins comprising IL-33 variants disclosed herein. In some embodiments, the multifunctional protein comprises an IL-33 variant and a second therapeutic polypeptide. In some embodiments, the second therapeutic polypeptide is a cytokine. In some embodiments, the IL-33 variant is associated with a half-life extension moiety. In some embodiments, the multifunctional protein comprises an IL-33 variant fused to a second therapeutic polypeptide in a fusion protein. In some embodiments, the fusion protein comprises a linker which links the IL-33 variant with the second therapeutic polypeptide.

In some embodiments, the cytokine activates the JAK/STAT pathway. Upon binding of the cytokine to its receptor, the receptor dimerizes and different types of receptor-associated Janus family tyrosine kinases (JAK) are activated which phosphorylate the receptor and lead to recruitment and activation of other kinases and transcription factors, such as those of the Signal Transducer and Activator of Transcription (STAT) family. This promotes the rapid translocation of these proteins to the nucleus and stimulation of downstream target gene transcription.

In some embodiments, the cytokine is selected from a group consisting of IL-2, IL-4, IL-6, IL-7, IL-10, IL-12, IL-13 and IL-15. In some embodiments, the cytokine is IL-2. In some embodiments, the cytokine is IL-4. In some embodiments, the cytokine is IL-6. In some embodiments, the cytokine is IL-7. In some embodiments, the cytokine is IL-10. In some embodiments, the cytokine is IL-12. In some embodiments, the cytokine is IL-13. In some embodiments, the cytokine is IL-15.

In some embodiments, the cytokine is an interferon. In some embodiments, the cytokine is an IFN-α. In some embodiments, the cytokine is an IFN-β. In some embodiments, the cytokine is an IFN-γ.

In some embodiments, the second therapeutic polypeptide is a Tumor Necrosis Factor (TNF). In some embodiments, the second therapeutic polypeptide is CD154, LT-B, TNF-a, TNF-B, 4-1BBL, APRIL, CD70, CD153, CD178, GITRL, LIGHT, OX40L, TALL-1, TRAIL, TWEAK or TRANCE.

In some embodiments, the second therapeutic factor is a TGF-β. In some embodiments, the second therapeutic factor is a TGF-β1. In some embodiments, the second therapeutic factor is a TGF-β2. In some embodiments, the second therapeutic factor is a TGF-β3.

In some embodiments, the second therapeutic polypeptide is an antibody. In some embodiments, the second therapeutic polypeptide is a proapoptotic agent. In some embodiments, the second therapeutic polypeptide is a targeting moiety that targets the IL-33 variant to a specific tissue location. In some embodiments, the second therapeutic polypeptide enhances expression of the IL-33 variant. In some embodiments, the second therapeutic polypeptide enhances half-life of the IL-33 variant.

In some embodiments, the second therapeutic polypeptide is an erythropoietin. In some embodiments, the second therapeutic polypeptide is a TPO. In some embodiments, the second therapeutic polypeptide is a Flt-3L. In some embodiments, the second therapeutic polypeptide is a stem cell factor (SCF). In some embodiments, the second therapeutic polypeptide is a M-CSF. In some embodiments, the second therapeutic polypeptide is a Macrophage Stimulating Protein. In some embodiments, the second therapeutic polypeptide is a Macrophage Stimulating Factor (MST-1).

In some embodiments, the second therapeutic polypeptide is interleukin-2 (IL-2).

### Interleukin-2 (IL-2)

In some embodiments of the multifunctional fusion protein, the interleukin-2 (IL-2) polypeptide is a wild-type IL-2 polypeptide. Interleukin-2 (IL2) is a Type 1 immunostimulatory cytokine for key immune cells including T cells and natural killer (NK) cells. Systemic IL-2 supplementation enhances immunity in a variety of diseases ranging from neoplasms to viral infection. However, its systemic use is restricted by its serious side effects and its efficacy may be limited by a low half-life in vivo of about 10 minutes. IL-2 signaling is mediated through interactions with a high affinity multi-subunit receptor complex. IL-2 is secreted as a single glycosylated polypeptide, and cleavage of a signal sequence is required for its activity. Structurally, IL-2 comprises a bundle of 4 helices (termed A-D), flanked by 2 shorter helices and several loop regions. Residues in helix A, and in the loop region between helices A and B, are important for receptor binding. Secondary structure analysis has suggested similarity to IL-4 and granulocyte-macrophage colony stimulating factor (GMCSF).

IL-2R is a heterotrimeric protein expressed on a variety of different immune cell types, including T cells, NK cells, eosinophils, and monocytes. This broad expression pattern provides a pleiotropic effect on the immune system and a high systemic toxicity of IL-2 treatments, which makes targeting IL-2R+ cells challenging.

IL2-R has three forms, generated by different combinations of three different IL-2R proteins: α (alpha), β (beta), and γ (gamma). These receptor chains assemble to generate the three different receptor forms: (1) the low affinity receptor, IL2Rα, which does not signal; (2) the intermediate affinity receptor (IL2Rβγ), composed of IL2Rβ and IL2Rγ, which is broadly expressed on CD4+ T cells, NK cells, eosinophils, and monocytes; and (3) the high affinity receptor (IL2Rαβγ), composed of IL2Rα, IL2Rβ, and IL2Rγ, which is expressed transiently on activated T cells and constitutively on Treg cells. Mutations in IL-2 can change the binding affinity of IL-2 to different IL-2R receptor forms. Thus, the present disclosure provides compounds that selectively activate and expand Tregs, for example ST2+Tregs, by synergistically increasing binding of IL-2 to its alpha receptor, and also including an IL-33 variants. An exemplary moiety includes, but is not limited to, an IL-2 variant comprising one or more mutations that modifies the binding relative to WT.

In some embodiments of the multifunctional fusion protein, the IL-2 polypeptide is a variant IL-2.

### IL-2 Variants

Described herein are various IL-2 variants used to expand proliferation and/or activity of ST2+ Treg cells. The IL-2 variants described herein are used to treat autoimmune disease.

In some embodiments, the IL-2 polypeptide is an IL-2 variant biased to IL2Rα, i.e., the invention provides, among other things, immunosuppressive IL-2 variants that have a higher affinity for IL-2Rα than wild-type IL-2. In some embodiments, IL-2 variants contain one or more mutations in positions of the IL-2 sequence that either contact IL-2Rα or alter the orientation of other positions contacting IL-2Rα, resulting in higher affinity for IL-2Rα as determined by methods known in the art, for example, immobilized receptors and Biacore surface plasmon resonance assays. The mutations may be in or near areas known to be in close proximity to IL-2Rα based on crystal structures.

In some embodiments, the invention provides immunosuppressive IL-2 variants that have a lower affinity for IL-2Rβ than wild-type IL-2.

Immunosuppressive IL-2 variants also include variants that demonstrate altered signaling through certain pathways activated by wild-type IL-2 via the IL-2R and result in preferential proliferation/survival/activation of ST2+ cells, e.g., ST2+ T-reg cells. Signaling molecules known to be phosphorylated upon activation of the IL-2R include, for example, STAT5, p38, ERK, SYK, LCK, AKT and mTOR.

In some embodiments, as used herein, IL-2 variants suitable for the present invention include any wild-type and modified IL-2 variants (e.g., IL-2 proteins with amino acid mutations, deletions, insertions, and/or fusion proteins) that retain substantial IL-2 biological activity. Typically, a recombinant IL-2 protein is produced using recombinant technology. However, IL-2 proteins (wild-type or modified) purified from natural resources or synthesized chemically can be used according to the present invention.

IL-2 variants (also referred to herein as “IL-2 muteins”) comprise a sequence of amino acids at least 70%, at least 75%, at least 80%, at least 85%, at least 90%, at least 91%, at least 92%, at least 93% at least 94%, at least 95%, at least 96%, at least 97%, at least 98%, at least 99% identical to wild-type IL-2. IL-2 variants further include a sequence of amino acids at least 70%, at least 75%, at least 80%, at least 85%, at least 90%, at least 91%, at least 92%, at least 93% at least 94%, at least 95%, at least 96%, at least 97%, at least 98%, at least 99% identical to a functional fragment of wild-type IL-2.

Variants may contain one or more substitutions, deletions, or insertions within the wild-type IL-2 amino acid sequence. Residues are designated herein by the one letter amino acid code followed by the IL-2 amino acid position. Substitutions are designated herein by the one letter amino acid code followed by the IL-2 amino acid position followed by the substituting one letter amino acid code.

In some embodiments, the present invention provides human interleukin-2 variants comprising at least one amino acid substitution that can selectively activate proliferation of regulatory T cells.

In some embodiments, the IL-2 variant comprises one or more amino acid substitutions selected from in relation to the wild-type IL-2 selected from a group consisting of T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, 192R, E95G, C125A, C125S, Q1261, Q126L, and Q126F. It is understood that any mutation can be combined with any other mutation in any order from the above list. It is understood that any number of mutations from the above list can be combined in any order. It is understood that every position described above may be mutated and replaced by an amino acid substitution as described above or an equivalent amino acid.

In some embodiments of the multifunctional fusion protein, the IL-2 polypeptide comprises at least one amino acid substitution selected from N88R, N88S, C125S, V69A and Q74P. In some embodiments, the IL-2 polypeptide comprises an N88R amino acid substitution. In some embodiments, the IL-2 polypeptide comprises an N88S amino acid substitution. In some embodiments, the IL-2 polypeptide comprises an C125S amino acid substitution. In some embodiments, the IL-2 polypeptide comprises an V69A amino acid substitution. In some embodiments, the IL-2 polypeptide comprises an Q74P amino acid substitution.

In some embodiments, the IL-2 polypeptide comprises 2, 3, 4, or 5 mutations selected from N88R, N88S, C125S, V69A and Q74P. In some embodiments, the IL-2 polypeptide comprises N88R and C125S mutations. In some embodiments, the IL-2 polypeptide comprises N88R and V69A mutations. In some embodiments, the IL-2 polypeptide comprises N88R and Q74P mutations. In some embodiments, the IL-2 polypeptide comprises N88S and C125S mutations. In some embodiments, the IL-2 polypeptide comprises N88S and V69A mutations. In some embodiments, the IL-2 polypeptide comprises N88S and Q74P mutations. In some embodiments, the IL-2 polypeptide comprises C125S and V69A mutations. In some embodiments, the IL-2 polypeptide comprises C125S and Q74P mutations. In some embodiments, the IL-2 polypeptide comprises V69A and Q74P mutations.

In some embodiments, the IL-2 polypeptide comprises N88R, C125S and Q74P mutations. In some embodiments, the IL-2 polypeptide comprises N88S, C125S and Q74P mutations. In some embodiments, the IL-2 polypeptide comprises N88R, V69A and Q74P mutations. In some embodiments, the IL-2 polypeptide comprises N88S, V69A and Q74P mutations. In some embodiments, the IL-2 polypeptide comprises N88R, C125S and V69A mutations. In some embodiments, the IL-2 polypeptide comprises N88S, C125S and V69A mutations.

In some embodiments, the IL-2 variant comprises the amino acid substitution N88R. In some embodiments, the IL-2 variant comprises the amino acid substitution N88S.

In some embodiments, the IL-2 variant comprises the amino acid substitutions of N88D and C125S.

In some embodiments, the IL-2 variant comprises the amino acid substitutions of V69A, Q47P, N88D and C125S.

In some embodiments, a suitable recombinant IL-2 variant has an in vivo half-life of or greater than about 1 minute, 2 minutes, 10 minutes, 15 minutes, 30 minutes, 60 minutes, 70 minutes, 80 minutes, 90 minutes, 100 minutes, 2 hours, 3 hours, 4 hours, 5 hours, 6 hours, 7 hours, 8 hours, 9 hours, 10 hours, 12 hours, or 24 hours. In some embodiments, a suitable recombinant IL-2 mutein or a recombinant IL-2 fusion protein has an in vivo half-life of or greater than about 24 hours, 30 hours, 36 hours, 42 hours, 48 hours, 54 hours, or 60 hours. In some embodiments, a recombinant IL-2 mutein has an in vivo half-life of between 0.5 and 24 hours, between 1 day and 10 days, between 1 day and 9 days, between 1 day and 8 days, between 1 day and 7 days, between 1 day and 6 days, or between 1 day and 5 days.

### Multifunctional IL-33 and IL-2 Fusion Protein

In some aspects, an IL-2 polypeptide is associated with an IL-33 variant in a multifunctional protein that synergistically facilitates a therapeutic effect, for example, by enhancing or increasing stability, potency and/or delivery of IL-2 protein, reducing or eliminating immunogenicity, or clearance, etc. The present invention provides multifunctional proteins comprising variants that preferentially expand Tregs over, for example, T effector or NK cells. In some embodiments, the multifunctional protein described herein is a fusion protein between an IL-2 domain and an IL-33 variant.

In some aspects, provided herein is a multifunctional fusion protein comprising: an IL-2 polypeptide, an IL-33 polypeptide, and a half-life extension moiety, wherein the half-life extension moiety is located at the N-terminus or C-terminus of the fusion protein. In some embodiments, provided herein is a multifunctional fusion protein comprising: an IL-2 polypeptide, an IL-33 polypeptide, and a half-life extension moiety, wherein the half-life extension moiety is located at the N-terminus of the fusion protein. In some embodiments, provided herein is a multifunctional fusion protein comprising: an IL-2 polypeptide, an IL-33 polypeptide, and a half-life extension moiety, wherein the half-life extension moiety is located at the C-terminus of the fusion protein.

In some aspects, provided herein is a multifunctional fusion protein comprising: an IL-2 polypeptide, an IL-33 polypeptide, and a half-life extension moiety, wherein the half-life extension moiety is not an Fc domain of IgG.

In some aspects, provided herein is a multifunctional fusion protein, wherein the IL-33 polypeptide is a variant wherein the variant has improved activity and/or manufacturability relative to wild-type IL-33. In some aspects, provided herein is a multifunctional fusion protein, wherein the IL-33 polypeptide is a variant wherein the variant has improved activity relative to wild-type IL-33. In some aspects, provided herein is a multifunctional fusion protein, wherein the IL-33 polypeptide is a variant wherein the variant has improved manufacturability relative to wild-type IL-33.

In some aspects, provided herein is a multifunctional fusion protein, comprising at least one amino acid substitution at N60, C97, C116, C121, or C148, and wherein the substitution at any one of the cysteines C97, C116, C121, or C148 is not a serine. In some embodiments, provided herein is a multifunctional fusion protein, comprising at least one amino acid substitution at N60. In some embodiments, provided herein is a multifunctional fusion protein, comprising at least one amino acid substitution at C97, wherein the substitution at C97 is not a serine. In some embodiments, provided herein is a multifunctional fusion protein, comprising at least one amino acid substitution at C116, wherein the substitution at C116 is not a serine. In some embodiments, provided herein is a multifunctional fusion protein, comprising at least one amino acid substitution at C121, wherein the substitution at C121 is not a serine. In some embodiments, provided herein is a multifunctional fusion protein, comprising at least one amino acid substitution at C148, wherein the substitution at C148 is not a serine.

In some embodiments, provided herein is a multifunctional fusion protein, comprising at least one amino acid substitution at N60 and C97, and wherein the substitution at C97 is not a serine. In some embodiments, provided herein is a multifunctional fusion protein, comprising at least one amino acid substitution at N60 and C116, and wherein the substitution at C116 is not a serine. In some embodiments, provided herein is a multifunctional fusion protein, comprising amino acid substitutions at N60 and C121, and wherein the substitution at C121 is not a serine. In some embodiments, provided herein is a multifunctional fusion protein, comprising amino acid substitutions at N60 and C148, and wherein the substitution at C148 is not a serine. In some embodiments, provided herein is a multifunctional fusion protein, comprising amino acid substitutions at C97 and C116, and wherein the substitution at C97 and C116 is not a serine. In some embodiments, provided herein is a multifunctional fusion protein, comprising amino acid substitutions at C97 and C121, and wherein the substitution at C97 and C121 is not a serine. In some embodiments, provided herein is a multifunctional fusion protein, comprising amino acid substitutions at C97 and C148, and wherein the substitution at C97 and C148 is not a serine. In some embodiments, provided herein is a multifunctional fusion protein, comprising amino acid substitutions at C116 and C121, and wherein the substitution at C116 and C121 is not a serine. In some embodiments, provided herein is a multifunctional fusion protein, comprising amino acid substitutions at C116 and C148, and wherein the substitution at C116 and C148 is not a serine. In some embodiments, provided herein is a multifunctional fusion protein, comprising amino acid substitutions at C121 and C148, and wherein the substitution at C121 and C148 is not a serine. In some embodiments, provided herein is a multifunctional fusion protein, comprising amino acid substitutions at C97 and C121, and wherein the substitution at C97 and C121 is not a serine.

In some embodiments, provided herein is a multifunctional fusion protein, comprising amino acid substitutions at C97, C116 and C121, and wherein the substitution at C97, C116 and C121 is not a serine. In some embodiments, provided herein is a multifunctional fusion protein, comprising amino acid substitutions at C97, C121 and C148, and wherein the substitution at C97, C121 and C148 is not a serine. In some embodiments, provided herein is a multifunctional fusion protein, comprising amino acid substitutions at C97, C116 and C148, and wherein the substitution at C97, C116 and C148 is not a serine. In some embodiments, provided herein is a multifunctional fusion protein, comprising amino acid substitutions at C116, C121 and C148, and wherein the substitution at C116, C121 and C148 is not a serine.

In some embodiments, the multifunctional fusion protein comprises an amino acid substitution at N60 and any one of C97, C116, C121, or C148. In some embodiments, the multifunctional fusion protein comprises amino acid substitutions at N60 and C97. In some embodiments, the multifunctional fusion protein comprises amino acid substitutions at N60 and C116. In some embodiments, the multifunctional fusion protein comprises amino acid substitutions at N60 and C121. In some embodiments, the multifunctional fusion protein comprises amino acid substitutions at N60 and C148.

In some embodiments, the multifunctional fusion protein comprises an amino acid substitution at C116F and an additional amino acid substitution at any one of C97, C121, or C148. In some embodiments, the multifunctional fusion protein comprises an amino acid substitution at C116F and an additional amino acid substitution at C97. In some embodiments, the multifunctional fusion protein comprises an amino acid substitution at C116F and an additional amino acid substitution at C121. In some embodiments, the multifunctional fusion protein comprises an amino acid substitution at C116F and an additional amino acid substitution at C148.

In some embodiments, the multifunctional fusion protein comprises at least one amino acid substitution selected from N60S, N60D, C97G, and C116F. In some embodiments, the multifunctional fusion protein comprises an amino acid substitution N60S. In some embodiments, the multifunctional fusion protein comprises an amino acid substitution N60D. In some embodiments, the multifunctional fusion protein comprises an amino acid substitution C97G. In some embodiments, the multifunctional fusion protein comprises an amino acid substitution C116F.

In some embodiments, the multifunctional fusion protein comprises an amino acid substitution at N60, which is N60S, N60T, N60Q, N60D or N60E.

In some embodiments, the multifunctional fusion protein comprises an amino acid substitution at C97, which is C97G, C97A or C97V.

In some embodiments, the multifunctional fusion protein comprises an amino acid substitution at C116, which is C116F or C116Y.

In some embodiments, the multifunctional fusion protein comprises an amino acid substitution at C121, which is C121S.

In some embodiments, the multifunctional fusion protein comprises an amino acid substitution at C148, which is C148G, C148S, C148Y or C148N.

In some embodiments, the multifunctional fusion protein comprises the amino acid substitutions of C97G and C116F.

In some embodiments, the multifunctional fusion protein comprises the amino acid substitutions of C97G, C116F, and C148G.

In some embodiments, the multifunctional fusion protein comprises the amino acid substitutions of C97G, C116F, C121S, and C148G.

In some embodiments of the multifunctional fusion protein, the IL-33 is truncated.

In some embodiments of the multifunctional fusion protein, the IL-33 polypeptide comprises the sequence of any one of SEQ ID NOs: 2-8, and SEQ ID NOs: 81-82.

In some aspects, provided herein is a multifunctional protein comprising: an IL-2 polypeptide, and an IL-33 variant that has improved activity and/or manufacturability as compared to human WT IL-33, wherein the IL-2 polypeptide is associated with the IL-33 variant, and wherein the multifunctional protein preferentially expands ST2+ Treg cells.

### Half-Life Extension Moiety

In some embodiments, a half-life extension moiety is fused to any one of IL-33, second polypeptide (e.g. IL-2) or fusion proteins described herein. In some embodiments of the multifunctional fusion protein, the half-life extension moiety is selected from the group consisting of monomethoxy poly-ethylene glycol (PEG), albumin, transferrin, Fc domain of IgG, highly sialylated peptide, XTEN, V-P-G-x-G (ELP), proline-alanine-serine (PAS) polymer, homo amino acid polymer (HAP) and gelatin-like protein (GLK). In some embodiments, the half-life extension moiety is a monomethoxy poly-ethylene glycol (PEG). In some embodiments, the half-life extension moiety is an albumin. In some embodiments, the half-life extension moiety is a transferrin. In some embodiments, the half-life extension moiety is an Fc domain of IgG. In some embodiments, the half-life extension moiety is a highly sialylated peptide. In some embodiments, the half-life extension moiety is XTEN. In some embodiments, the half-life extension moiety is V-P-G-x-G (ELP). In some embodiments, the half-life extension moiety is a proline-alanine-serine (PAS) polymer. In some embodiments, the half-life extension moiety is a homo amino acid polymer (HAP). In some embodiments, the half-life extension moiety is a gelatin-like protein (GLK).

In some embodiments, the PEG is linear chain or branched chain. In some embodiments, the PEG is linear chain. In some embodiments, the PEG is branched chain.

In some embodiments, the albumin is a human serum albumin (HSA). In some embodiments, the half-life extension moiety is a human albumin binding domain. In some embodiments, the half-life extension moiety is a human albumin binding domain QMP. In some embodiments, the half-life extension moiety is a human albumin binding domain DARP.

In some embodiments, the human albumin binding domain comprises an amino acid sequence 100% identical to SEQ ID NO: 55. In some embodiments, the human albumin binding domain comprises an amino acid sequence at least 85% identical to SEQ ID NO: 55. In some embodiments, the human albumin binding domain comprises an amino acid sequence at least 90% identical to SEQ ID NO: 55. In some embodiments, the human albumin binding domain comprises an amino acid sequence at least 95% identical to SEQ ID NO: 55. In some embodiments, the human albumin binding domain comprises an amino acid sequence at least 99% identical to SEQ ID NO: 55.

In some embodiments, the human albumin binding domain QMP comprises an amino acid sequence 100% identical to SEQ ID NO: 56. In some embodiments, the human albumin binding domain QMP comprises an amino acid sequence at least 85% identical to SEQ ID NO: 56. In some embodiments, the human albumin binding domain QMP comprises an amino acid sequence at least 90% identical to SEQ ID NO: 56. In some embodiments, the human albumin binding domain QMP comprises an amino acid sequence at least 95% identical to SEQ ID NO: 56. In some embodiments, the human albumin binding domain QMP comprises an amino acid sequence at least 99% identical to SEQ ID NO: 56.

In some embodiments, the human albumin binding domain DARP comprises an amino acid sequence 100% identical to SEQ ID NO: 57. In some embodiments, the human albumin binding domain DARP comprises an amino acid sequence at least 85% identical to SEQ ID NO: 57. In some embodiments, the human albumin binding domain DARP comprises an amino acid sequence at least 90% identical to SEQ ID NO: 57. In some embodiments, the human albumin binding domain DARP comprises an amino acid sequence at least 95% identical to SEQ ID NO: 57. In some embodiments, the human albumin binding domain DARP comprises an amino acid sequence at least 99% identical to SEQ ID NO: 57.

In some embodiments, the highly sialylated peptide is a carboxy terminal peptide (CTP) consisting of the sequence of FQSSSS*

### KAPPPS*LPSPS*RLPGPS*DTPILPQ (SEQ ID NO: 51).

In some embodiments, the homo amino acid polymer consists of glycine.

In some embodiments, the Fc domain comprises at least 90% identity to SEQ ID NO: 54. In some embodiments, the Fc domain comprises 90% identity to SEQ ID NO: 54. In some embodiments, the Fc domain comprises 91% identity to SEQ ID NO: 54. In some embodiments, the Fc domain comprises 92% identity to SEQ ID NO: 54. In some embodiments, the Fc domain comprises 93% identity to SEQ ID NO: 54. In some embodiments, the Fc domain comprises 94% identity to SEQ ID NO: 54. In some embodiments, the Fc domain comprises 95% identity to SEQ ID NO: 54. In some embodiments, the Fc domain comprises 96% identity to SEQ ID NO: 54. In some embodiments, the Fc domain comprises 97% identity to SEQ ID NO: 54. In some embodiments, the Fc domain comprises 98% identity to SEQ ID NO: 54. In some embodiments, the Fc domain comprises 99% identity to SEQ ID NO: 54. In some embodiments, the Fc domain comprises greater than 99% identity to SEQ ID NO: 54.

In some embodiments of the multifunctional fusion protein, wherein the Fc domain comprises 100% identity to the wild-type sequence of SEQ ID NO: 54.

In some embodiments of the multifunctional fusion protein, wherein the Fc domain comprises one or more mutations that reduce effector function.

In some embodiments of the multifunctional fusion protein, wherein the one or more mutations are selected from N297A, N297Q, N297G, L234A, L235E, G237A, P238S, H268Q, H268A, V309L, A330S and P331S. In some embodiments, the one or more mutations are N297A. In some embodiments, the mutation is N297Q. In some embodiments, the mutation is N297G. In some embodiments, the mutation is L234A. In some embodiments, the mutation is L235E. In some embodiments, the mutation is G237A. In some embodiments, the mutation is P238S. In some embodiments, the mutation is H268Q. In some embodiments, the mutation is H268A. In some embodiments, the mutation is V309L. In some embodiments, the mutation is A330S. In some embodiments, the mutation is P331S.

In some embodiments, the multifunctional fusion protein comprises two mutations selected from N297A, N297Q, N297G, L234A, L235E, G237A, P238S, H268Q, H268A, V309L, A330S and P331S. It is to be understood by a skilled person that this includes any and all permutations and combinations of two mutations from the above list selected in any order.

In some embodiments, the multifunctional fusion protein comprises 3 mutations selected from N297A, N297Q, N297G, L234A, L235E, G237A, P238S, H268Q, H268A, V309L, A330S and P331S. It is to be understood by a skilled person that this includes any and all permutations and combinations of 3 mutations from the above list selected in any order.

In some embodiments, the multifunctional fusion protein comprises 4 mutations selected from N297A, N297Q, N297G, L234A, L235E, G237A, P238S, H268Q, H268A, V309L, A330S and P331S. It is to be understood by a skilled person that this includes any and all permutations and combinations of 4 mutations from the above list selected in any order.

In some embodiments, the multifunctional fusion protein comprises 5 mutations selected from N297A, N297Q, N297G, L234A, L235E, G237A, P238S, H268Q, H268A, V309L, A330S and P331S. It is to be understood by a skilled person that this includes any and all permutations and combinations of 5 mutations from the above list selected in any order.

In some embodiments, the multifunctional fusion protein comprises 6 mutations selected from N297A, N297Q, N297G, L234A, L235E, G237A, P238S, H268Q, H268A, V309L, A330S and P331S. It is to be understood by a skilled person that this includes any and all permutations and combinations of 6 mutations from the above list selected in any order.

In some embodiments, the multifunctional fusion protein comprises 7 mutations selected from N297A, N297Q, N297G, L234A, L235E, G237A, P238S, H268Q, H268A, V309L, A330S and P331S. It is to be understood by a skilled person that this includes any and all permutations and combinations of 7 mutations from the above list selected in any order.

In some embodiments, the multifunctional fusion protein comprises 8 mutations selected from N297A, N297Q, N297G, L234A, L235E, G237A, P238S, H268Q, H268A, V309L, A330S and P331S. It is to be understood by a skilled person that this includes any and all permutations and combinations of 8 mutations from the above list selected in any order.

In some embodiments, the multifunctional fusion protein comprises 9 mutations selected from N297A, N297Q, N297G, L234A, L235E, G237A, P238S, H268Q, H268A, V309L, A330S and P331S. It is to be understood by a skilled person that this includes any and all permutations and combinations of 9 mutations from the above list selected in any order.

In some embodiments, the multifunctional fusion protein comprises 10 mutations selected from N297A, N297Q, N297G, L234A, L235E, G237A, P238S, H268Q, H268A, V309L, A330S and P331S. It is to be understood by a skilled person that this includes any and all permutations and combinations of 10 mutations from the above list selected in any order.

In some embodiments, the multifunctional fusion protein comprises 11 mutations selected from N297A, N297Q, N297G, L234A, L235E, G237A, P238S, H268Q, H268A, V309L, A330S and P331S. It is to be understood by a skilled person that this includes any and all permutations and combinations of 11 mutations from the above list selected in any order. In some embodiments, the multifunctional fusion protein comprises 11 mutations selected from N297Q, N297G, L234A, L235E, G237A, P238S, H268Q, H268A, V309L, A330S and P331S. In some embodiments, the multifunctional fusion protein comprises 11 mutations selected from N297A, N297G, L234A, L235E, G237A, P238S, H268Q, H268A, V309L, A330S and P331S. In some embodiments, the multifunctional fusion protein comprises 11 mutations selected from N297A, N297Q, L234A, L235E, G237A, P238S, H268Q, H268A, V309L, A330S and P331S. In some embodiments, the multifunctional fusion protein comprises 11 mutations selected from N297A, N297Q, N297G, L235E, G237A, P238S, H268Q, H268A, V309L, A330S and P331S. In some embodiments, the multifunctional fusion protein comprises 11 mutations selected from N297A, N297Q, N297G, L234A, G237A, P238S, H268Q, H268A, V309L, A330S and P331S. In some embodiments, the multifunctional fusion protein comprises 11 mutations selected from N297A, N297Q, N297G, L234A, L235E, P238S, H268Q, H268A, V309L, A330S and P331S. In some embodiments, the multifunctional fusion protein comprises 11 mutations selected from N297A, N297Q, N297G, L234A, L235E, G237A, H268Q, H268A, V309L, A330S and P331S. In some embodiments, the multifunctional fusion protein comprises 11 mutations selected from N297A, N297Q, N297G, L234A, L235E, G237A, P238S, H268A, V309L, A330S and P331S. In some embodiments, the multifunctional fusion protein comprises 11 mutations selected from N297A, N297Q, N297G, L234A, L235E, G237A, P238S, H268Q, V309L, A330S and P331S. In some embodiments, the multifunctional fusion protein comprises 11 mutations selected from N297A, N297Q, N297G, L234A, L235E, G237A, P238S, H268Q, H268A, A330S and P331S. In some embodiments, the multifunctional fusion protein comprises 11 mutations selected from N297A, N297Q, N297G, L234A, L235E, G237A, P238S, H268Q, H268A, V309L and A330S.

In some embodiments, the multifunctional fusion protein comprises 12 mutations comprising N297A, N297Q, N297G, L234A, L235E, G237A, P238S, H268Q, H268A, V309L, A330S and P331S.

In some embodiments of the multifunctional fusion protein, the IL-2 and IL-33 polypeptides are 10 to 100 angstrom units (Å) apart. In some embodiments of the multifunctional fusion protein, the IL-2 and IL-33 polypeptides are 10 angstrom units (Å) apart. In some embodiments of the multifunctional fusion protein, the IL-2 and IL-33 polypeptides are 20 angstrom units (Å) apart. In some embodiments of the multifunctional fusion protein, the IL-2 and IL-33 polypeptides are 30 angstrom units (Å) apart. In some embodiments of the multifunctional fusion protein, the IL-2 and IL-33 polypeptides are 40 angstrom units (Å) apart. In some embodiments of the multifunctional fusion protein, the IL-2 and IL-33 polypeptides are 50 angstrom units (Å) apart. In some embodiments of the multifunctional fusion protein, the IL-2 and IL-33 polypeptides are 60 angstrom units (Å) apart. In some embodiments of the multifunctional fusion protein, the IL-2 and IL-33 polypeptides are 70 angstrom units (Å) apart. In some embodiments of the multifunctional fusion protein, the IL-2 and IL-33 polypeptides are 80 angstrom units (Å) apart. In some embodiments of the multifunctional fusion protein, the IL-2 and IL-33 polypeptides are 90 angstrom units (A) apart. In some embodiments of the multifunctional fusion protein, the IL-2 and IL-33 polypeptides are 100 angstrom units (Å) apart.

### Linker

In some embodiments, the multifunctional protein further comprises a linker. Each domain of the fusion protein may be linked by a linker, for example, a non-immunogenic linker that is long enough to allow free rotation of individual cytokines.

In some embodiments of the multifunctional fusion protein, the linker is a peptide linker or a chemical linker. In some embodiments, the linker is a peptide linker. In some embodiments, the linker is a chemical linker.

In some embodiments, the linker comprises neutral amino acids. In some embodiments, the linker comprises a sequence of GGGGS (SEQ ID NO: 29) or GGGGSGGGGSGGGGS (SEQ ID NO: 30).

In some embodiments, the linker comprises a sequence of GGGGS (SEQ ID NO: 29).

In some embodiments, the linker comprises (GGGGS) n, wherein n is any number from 1 to 10. In some embodiments, the linker comprises (Gly) 8. In some embodiments, the linker comprises (GS) n, wherein n is any number from 1 to 10. In some embodiments, the linker comprises (GS) n (n=2-10).

In some embodiments, the linker comprises (GGSGGGS) n, wherein n is any number from 1 to 10, for example, GGSGGSGGS (SEQ ID NO: 36), n=3.

In some embodiments, the linker comprises EAAAK (SEQ ID NO: 32). In some embodiments, the linker comprises (EAAAK) n, wherein n is any number from 1 to 5. In some embodiments, the linker comprises (GGGGS) n (EAAAK) n, wherein n is any number from 1 to 3, for example SEQ ID NO: 42, n=1.

In some embodiments, the linker comprises a sequence of GGGGSGGGGSGGGGS (SEQ ID NO: 30).

In some embodiments of the multifunctional protein or the fusion protein, wherein the peptide linker is selected from GGGGS (SEQ ID NO: 29; n repeats of SEQ ID NO: 29, n=1-5), GGGGSGGGGSGGGGS (SEQ ID NO: 30), GGSGGGS (SEQ ID NO:31), EAAAK (SEQ ID NO: 32; n repeats of SEQ ID NO: 32, n=1-3), GGGGGGGG (SEQ ID NO: 33), GSGSGSGSGS (SEQ ID NO: 34), GSGSGSGSGSGSGSGSGSGS (SEQ ID NO: 35), GGSGGSGGS (SEQ ID NO: 36), GSGGS (SEQ ID NO: 37), GSSGS (SEQ ID NO: 38), a SEG-linker, a GSAT (SEQ ID NO: 39), SSSSGSSSSG (SEQ ID NO: 40), a flexible 22 amino acid linker LEGSGQGPGSGQGSGSPGSGQG (SEQ ID NO: 41), and GGGGSEAAAK (SEQ ID NO: 42, n=1-3), EGKSSGSGSESKST (SEQ ID NO: 44), GGGGSLVPRGSGGGGS (SEQ ID NO: 45), KESGSVSSEQLAQFRSLD (SEQ ID NO: 46), GGGSEGGGSEGGGSEGGG (SEQ ID NO: 47) or rigid peptide linkers PAPAP (SEQ ID NO: 48), (Ala-Pro) n, AEAAAKEAAAKA (SEQ ID NO: 49), AEAAAKEAAAKEAAAKEAAAKALEAEAAAKEAAAKEAAAKEAAAKA (SEQ ID NO: 50).

In some embodiments, the linker may be a cleavable peptide linker, such as a 2A self-cleaving peptide. Exemplary 2A peptides include T2A, P2A, F2A and E2A.

In some embodiments, the multifunctional fusion protein further comprises a tag at the N-terminus or C-terminus.

The tag herein is any affinity tag known in the art suitable for recombinant protein purification. In some embodiments, the tag is a Flag tag. In some embodiments, the tag is a His tag. In some embodiments, the tag is an HA tag. In some embodiments, the tag is a CBP tag. In some embodiments, the tag is a MBP tag. In some embodiments, the tag is a GST tag. In some embodiments, the tag is a Myc tag. In some embodiments, the tag is a SUMO tag. In some embodiments, the tag is a TAP tag. In some embodiments, the tag is a V5 tag. In some embodiments, the tag is a TRX tag.

In some embodiments, the multifunctional fusion protein comprises the sequence of any one of SEQ ID NO: 58-79.

In some embodiments, the fusion protein is lyophilized.

In some embodiments, provided herein is a polynucleotide encoding the IL-33 polypeptide or the multifunctional fusion protein described herein.

### Gene Therapy

In some embodiments, provided herein is a gene therapy vector comprising the polynucleotide described herein.

In some embodiments, the gene therapy vector is a viral vector or a non-viral vector.

In some embodiments, the gene therapy vector is a viral vector, including, for example and without limitation, adeno-associated virus vectors (AAV), lentiviral vectors, and adenovirus vectors. In some embodiments, the viral vector is without limitation, helper-dependent adenoviral, hybrid adenoviral, herpes simplex virus, poxvirus, Epstein-Barr virus, vaccinia virus, and human cytomegalovirus vector, including recombinant versions thereof.

Gene therapy vectors provide a means for delivering nucleic acids into a broad range of cells, including dividing and non-dividing cells. Gene therapy vectors can be employed to deliver a nucleic acid of interest to a cell in vitro, e.g., for ex vivo gene therapy. The vectors are additionally useful in a method of delivering a nucleic acid to a subject in need thereof, e.g., to express an immunogenic or therapeutic polypeptide or a functional RNA, thus the polypeptide or functional RNA can be produced in vivo in the subject.

Gene therapy vectors are used to treat and/or prevent any disease state for which it is beneficial to deliver a therapeutic polypeptide or functional nucleic acid.

The nucleic acid delivery vectors may also be employed to provide a functional nucleic acid to a cell in vitro or in vivo. Expression of the functional nucleic acid in the cell, for example, can diminish expression of a particular target protein by the cell, e.g., a downstream target in a signaling pathway. Accordingly, functional nucleic acid can be administered to decrease expression of a particular protein in a subject in need thereof. The nucleic acid delivery vectors can also be used for the purpose of evaluating safety (spread, toxicity, immunogenicity, etc.).

### Delivery

The nucleic acids described herein encoding IL-2 and IL-33 cytokines, can be delivered to a cell of interest by various delivery systems such as vectors, e.g., plasmids and delivery vectors.

In some embodiments, the cytokine variants and/or multifunctional fusion proteins can be delivered by nanoparticles, which can be organic or inorganic. Nanoparticles are well known in the art. Any suitable nanoparticle design can be used to deliver cytokines or nucleic acids encoding cytokines. For instance, organic (e.g., lipid and/or polymer) nanoparticles can be suitable for use as delivery vehicles in certain embodiments of this disclosure. Exemplary lipids for use in nanoparticle formulations, and/or gene transfer are shown in Table 2 (below).

In some embodiments, provided herein is a microparticle comprising the polynucleotide or the multifunctional fusion protein described herein. In some embodiments, the microparticle is a liposome, extracellular vesicle or a lipid nanoparticle. In some embodiments, microparticles comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids. In some embodiments, microparticles are used for a controlled release of therapeutic agent.

In some embodiments, delivery is by any one of the means listed in Table 1 below, as non-limiting examples.

TABLE 1
Modes of delivery of Recombinant IL-33, IL-2 or Fusions Thereof

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| | Engineered | YES | Transient | NO | Nucleic Acids |
| | Lactobacilli | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

Any suitable promoter can be used to drive expression of cytokines IL-33 and IL-2. A promoter used to drive expression of cytokines can include AAV ITR, which eliminates the need for an additional promoter element in a compact vector. ITR activity is relatively weak, so it can be used to reduce potential toxicity due to over expression of the chosen nuclease. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For osteoblasts, suitable promoters can include OG-2.

In some cases, separate promoters drive expression of the IL-2 and IL-33 within the same nucleic acid molecule. For instance, a vector or viral vector can comprise a first promoter operably linked to a nucleic acid encoding IL-2 and a second promoter operably linked to a nucleic acid encoding IL-33. Promoters include Pol III promoters such as U6 or H1.

IL-33 variants or multifunctional fusion proteins comprising IL-33 and IL-2 can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome. An AAV can be AAV1, AAV2, AAV4, AAV5, AAV8, AAV9 or any combination thereof. One can select the type of AAV with regard to the cells to be targeted; e.g., one can select AAV serotypes 1, 2, 5, 8, 9 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types. In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example an IL-33 or IL-2 encoding mRNA, or an mRNA encoding the multifunctional fusion protein can be delivered in the form of RNA. mRNA can be generated using in vitro transcription, for example, from a PCR cassette containing the following elements: T7 promoter, optional kozak sequence, nuclease sequence, and 3′ UTR such as a 3′ UTR from beta globin-polyA tail. The cassette can be used for transcription by T7 polymerase.

To enhance expression and reduce possible toxicity, the nucleotide sequence can be modified to include one or more modified nucleoside e.g., using pseudo-U or 5-Methyl-C.

The system can comprise one or more different vectors. In an aspect, the IL-33 and IL-2 nucleic acids are codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/(visited Jul. 9, 2002), and these tables can be adapted in a number of ways. See, Nakamura, “., et al. “Codon usage tabulated from the international DNA sequence databases: status for th” year 2000″ Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Engineered Host Cell

The disclosure in some embodiments provides a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the IL-2 and IL-33 of the present disclosure can be such that the cell and progeny of the cell are altered for improved cytokine expression or other desired cellular output. The modification introduced to the cell by the methods of the present disclosure can be such that the cell and progeny of the cell include an alteration that changes the biologic product produced. In some embodiments, the engineered host cell is used for manufacturing recombinant protein. In some embodiments, the engineered host cell is used for ex vivo therapy.

In some embodiments, provided herein is an engineered host cell comprising the polynucleotide or the vector described herein.

In some embodiments, the cell is a mammalian cell. In some embodiments, the cell is a Chinese hamster ovary (CHO) cell.

In some embodiments, the cell is a bacterial cell. In some embodiments, the cell is an E. coli cell.

In some embodiments, the cell is an insect cell. In some embodiments, the cell is a yeast cell.

### Manufacturing Methods

The IL-33 variants, IL-2 variants and/or fusion proteins comprising IL-33, IL-2 and a half-life extension moiety described herein can be produced using any suitable method known in the art. Such methods include, for example, constructing a DNA sequence encoding the IL-33 and/or IL-2 variant and expressing those sequences in a suitably transformed host. This method will produce the recombinant variant of this invention. However, the variants may also be produced by chemical synthesis or a combination of chemical synthesis and recombinant DNA technology. Batch-wise production or perfusion production methods are known in the art. See Freshey, R. I. (ed), 3 “Animal Cell Culture: A Practical Approach,” 2nd ed., 1992, IRL Press. Oxford, England; Mather, J. P. “Laboratory Scaleup of Cell Cultures (0.5-50 liters),” Methods Cell Biolog 57:219-527 (1998); Hu, W. S., and Aunins, J. G., “Large-scale Mammalian Cell Culture,” Curr Opin Biotechnol 8:148-153 (1997); Konstantinov, K. B., Tsai, Y., Moles, D., Matanguihan, R., “Control of long-term perfusion Chinese hamster ovary cell culture by glucose auxostat.,” Biotechnol Prag 12:100-109 (1996).

In some embodiments of producing the IL-33 variants and/or IL-2 variants described herein, a DNA sequence is constructed by isolating or synthesizing a DNA sequence encoding the wild type IL-33 or wild-type IL-2 and then changing one or more codons by site specific mutagenesis. See, e.g., Mark et. al., “Site-specific Mutagenesis of The Human Fibroblast Interferon Gene”, Proc. Natl. Acad. Sci. USA 81, pp. 5662-66 (1984); and U.S. Pat. No. 4,588,585, incorporated herein by reference. Various mutations and manners of creating same are known in the art and include, for example, amino acid and/or nucleic acid deletions, insertions, substitutions and/or fusions.

Another method of constructing a DNA sequence encoding the IL-33 variant and/or IL-2 variant would be chemical synthesis. This for example includes direct synthesis of a peptide by chemical means of the protein sequence encoding for an IL-33 variant or IL-2 variant exhibiting the properties described herein. This method may incorporate both natural and unnatural amino acids. Alternatively, a gene which encodes the desired IL-33 and/or IL-2 variant may be synthesized by chemical means using an oligonucleotide synthesizer. In some embodiments, such oligonucleotides are designed based on the amino acid sequence of the desired IL-33 and/or IL-2 variant, and selecting those codons that are favored in the host cell in which the recombinant variant will be produced. In this regard, it is well recognized that the genetic code is degenerate—that an amino acid may be coded for by more than one codon. For example, Phe (F) is coded for by two codons, TTC or TTT, Tyr (Y) is coded for by TAC or TAT and his (H) is coded for by CAC or CAT. Trp (W) is coded for by a single codon, TGG. Accordingly, it will be appreciated that for a given DNA sequence encoding a particular IL-33 and/or IL-2 variant, there will be many DNA degenerate sequences that will code for that IL-33 and/or IL-2 variant.

The DNA sequence encoding the IL-33 and/or IL-2 variant, whether prepared by site directed mutagenesis, chemical synthesis or other methods, may or may not also include DNA sequences that encode a signal sequence. In some embodiments, such signal sequence, if present, is one recognized by the cell chosen for expression of the IL-33 and/or IL-2 variant. It may be prokaryotic, eukaryotic or a combination of the two. It may also be the signal sequence of native IL-33 or IL-2. The inclusion of a signal sequence depends on whether it is desired to secrete the IL-33 or IL-2 variant from the recombinant cells in which it is made. In some embodiments, if the chosen cells are prokaryotic, the DNA sequence does not encode a signal sequence. In some embodiments, if the chosen cells are eukaryotic, a signal sequence is encoded and may have the wild-type IL-33 or IL-2 signal sequence.

Standard methods may be applied to synthesize a gene encoding an IL-33 or IL-2 variant. For example, the complete amino acid sequence may be used to construct a back translated gene. A DNA oligomer containing a nucleotide sequence coding for an IL-33 or IL-2 variant may be synthesized. For example, several small oligonucleotides coding for portions of the desired polypeptide may be synthesized and then ligated. The individual oligonucleotides may contain 5′ or 3′ overhangs for complementary assembly.

Once assembled (by synthesis, site-directed mutagenesis or another method), the DNA sequences encoding an IL-33 or IL-2 variant will be inserted into an expression vector and operatively linked to an expression control sequence appropriate for expression of the IL-33 or IL-2 variant in the desired transformed host. Proper assembly may be confirmed by nucleotide sequencing, restriction mapping, and expression of a biologically active polypeptide in a suitable host. As is known in the art, in order to obtain high expression levels of a transfected gene in a host, the gene is operatively linked to transcriptional and translational expression control sequences that are functional in the chosen expression host. The choice of expression control sequence and expression vector will depend upon the choice of host. A wide variety of expression host/vector combinations may be employed.

Any suitable host may be used to produce the IL-33 or IL-2 variant, including bacteria, fungi (including yeasts), plant, insect, mammal, or other appropriate animal cells or cell lines, as well as transgenic animals or plants. These hosts may include well known eukaryotic and prokaryotic hosts, such as strains of E. coli, Pseudomonas, Bacillus, Streptomyces, fungi, yeast, insect cells such as Spodoptera frugiperda (Sf9), animal cells such as Chinese hamster ovary (CHO) and mouse cells such as NS/0, African green monkey cells such as COS 1, COS 7, BSC 1, BSC 40, and BNT 10, and human cells, as well as plant cells in tissue culture. In some embodiments, for animal cell expression, CHO cells and COS 7 cells in cultures and the CHO cell line CHO (DHFR−) or the HKB line may be used.

It should be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host cell is considered because the vector must replicate in it. The vector copy number, the ability to control that copy number, and the expression of any other proteins encoded by the vector, such as antibiotic markers, may also be considered. For example, in some embodiments, vectors for use in this invention include those that allow the DNA encoding the IL-33 and/or IL-2 variants to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) or glutamine synthetase (“GS”) amplification (see, e.g., U.S. Pat. No. 5,122,464 and European published application 338,841).

The IL-33 and/or IL-2 variants may be glycosylated or unglycosylated depending on the host organism used to produce the variant. In some embodiments, when bacteria are chosen as the host, then the IL-33 and/or IL-2 variants produced will be unglycosylated. In some embodiments, eukaryotic cells will glycosylate the IL-33 and/or IL-2 variants. The IL-33 and/or IL-2 variants produced by the transformed host can be purified according to any suitable method. Various methods are known for purifying IL-33 and/or IL-2. See, e.g., Current Protocols in Protein Science, Vol. 2. Eds: John E. Coligan, Ben M. Dunn, Hidde L. Ploehg, David W. Speicher, Paul T. Wingfield, Unit 6.5 (Copyright 1997, John Wiley and Sons, Inc).

### Pharmaceutical Composition

In some embodiments, provided herein is a pharmaceutical composition comprising the multifunctional fusion protein described herein.

Aspects of the present disclosure relate to pharmaceutical compositions comprising IL-2 and/or IL-33 variants or nucleic acids or vectors encoding the same. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds). As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et ah, 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6:1438-47). Positively charged lipids such as N-[1-(2,3-dioleoyloxi) propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent, i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the IL-33 and IL-2 and/or multifunctional proteins or nucleic acids or RNA encoding the same are provided as part of a pharmaceutical composition. In some embodiments, the pharmaceutical composition comprises any of the fusion proteins provided herein (e.g., fusion of IL-2 and IL-3 polypeptides, including variants thereof, the fusion comprising, for example, a linker and half-life extension moiety). In some embodiments, the pharmaceutical composition comprises any of the complexes provided herein. In some embodiments, the pharmaceutical composition comprises a nucleic acid encoding IL-2 and IL-33. In some embodiments, the pharmaceutical composition comprises a messenger RNA encoding IL-2 and IL-33. In some embodiments, the pharmaceutical composition comprises a circular messenger RNA encoding IL-2 and IL-33. Pharmaceutical compositions can optionally comprise one or more additional therapeutically active substances.

In some embodiments, provided herein is a method of treating autoimmune disease, the method comprising administering to a subject in need thereof a therapeutically effective dose of the multifunctional fusion protein or the pharmaceutical composition or the engineered cell described herein.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Treating Autoimmune Disease or Disorder

In some embodiments, provided herein is a method of treating an autoimmune disease, the method comprising administering to a subject in need thereof a therapeutically effective dose of a multifunctional fusion protein of an IL-2 polypeptide, an IL-33 polypeptide, and a half-life extension moiety, wherein the half-life extension moiety is located at the N-terminus or C-terminus of the fusion protein.

In some embodiments, administration of the composition removes or reduces the effect of antibodies in a subject. In some embodiments, the subject has an autoimmune disease. In some embodiments, the antibodies are pathogenic autoantibodies. In some embodiments, administration of the composition is used to treat an autoimmune disorder.

Autoimmune diseases, disorders, or conditions may be amenable to treatment with or may be prevented by administration of IL-33 and IL-2 cytokines that synergistically promote ST2+ Treg proliferation and/or activity in a subject. In some embodiments, one or more IL-33 and IL-2 variants described herein are used to treat an autoimmune disease, disorder or condition. ST2+ Tregs are found in tissues such as muscle, visceral adipose, colon, and lung, and possess immunoregulatory and tissue repair functions.

In some embodiments, the ST2+ cells are ST2+ T regulatory cells, tissue resident ST2⁺ cells or a combination of tissue resident ST2⁺, CD25^{bright} cells and CD25^{bright}cells. In some embodiments, the ST2+ cells are ST2⁺ T regulatory cells. In some embodiments, the ST2+ cells are tissue resident ST2⁺ cells. In some embodiments, the ST2+ cells comprise a combination of tissue resident ST2⁺, CD25^{bright} cells and CD25^{bright}.

In some embodiments, the proliferation and/or activation of ST2+ T regulatory cells is at least greater than 100-fold relative to a control. In some embodiments, the proliferation of ST2+ T regulatory cells is at least greater than 100-fold relative to a control. In some embodiments, the activation of ST2+ T regulatory cells is at least greater than 100-fold relative to a control.

In some embodiments, the administration leads to coexpression of IL-2 and IL-33 in the same tissue.

In some embodiments, IL-2 and IL-33 synergistically show increased potency and/or increased avidity relative to IL-2 or IL-33 alone. In some embodiments, IL-2 and IL-33 synergistically show increased potency relative to IL-2 or IL-33 alone. In some embodiments, IL-2 and IL-33 synergistically show increased avidity relative to IL-2 or IL-33 alone.

In some embodiments, administration increases local immune tolerance in a tissue microenvironment. In some embodiments, the immune cells are ST2+ cells, Foxp3-ST2⁺ and Foxp3-ST2-Tr1 cells, Th3 cells, ILC2, macrophages, MDSCs, regulatory B cells, B1B cells or tolerogenic dendritic cells. In some embodiments, the immune cells are ST2+ cells. In some embodiments, the immune cells are Foxp3-ST2+ and Foxp3-ST2-Tr1 cells. In some embodiments, the immune cells are Th3 cells. In some embodiments, the immune cells are ILC2. In some embodiments, the immune cells are macrophages. In some embodiments, the immune cells are MDSCs. In some embodiments, the immune cells are regulatory B cells. In some embodiments, the immune cells are B1B cells. In some embodiments, the immune cells are tolerogenic dendritic cells.

Such diseases, disorders, and conditions that may be diminished in onset and/or severity include, but are not limited to, inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, poloymyalgia rheumatica, sarcoidosis, scleroderma, sclerosis, primary biliary sclerosis, sclerosing cholangitis, Sjogren's syndrome, atopic dermatitis, IgA nephropathy, membranous nephropathy, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple sclerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, transplantation rejection, and the like. In specific embodiments, pharmaceutical compositions comprising a therapeutically effective amount of an IL-33 and IL-2 fusion protein are provided.

In some embodiments, the disease is selected from a group consisting of MS, lupus, ankylosing spondylitis, arthritis, colitis, Type I diabetes, mitigate severity of inflammatory disease, Crohn's disease, heart disease, mitigate complications from immune response in pregnancy, mitigate complications from graft-versus-host disease (GVHD), mitigate severity of allergies, reduce rejection of HSC or allogeneic solid organ transplant, depression, ALS and/or myasthenia gravis.

The term “treatment” encompasses alleviation or prevention of at least one symptom or other aspect of a disorder, or reduction of disease severity, and the like. An ST2+ T-reg-selective IL-33 variant or fusion protein comprising IL-33 variant and IL-2 together with a half-life extension moiety need not effect a complete cure, or eradicate every symptom or manifestation of a disease, to constitute a viable therapeutic agent. As is recognized in the pertinent field, drugs employed as therapeutic agents may reduce the severity of a given disease state but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the invention is directed to a method comprising administering to a patient an ST2+ T-reg-selective multifunctional protein comprising IL-33 variant, IL-2 polypeptide and a half-life extension moiety in an amount and for a time sufficient to prevent or treat i.e., induce a sustained improvement over baseline of an indicator that reflects the severity of the particular disorder.

In some embodiments, provided herein is a method of treating autoimmune disease, the method comprising administering to a subject in need thereof a therapeutically effective dose of the multifunctional fusion protein, the pharmaceutical composition or the engineered cell described herein. In some embodiments, provided herein is a method of treating an autoimmune disease, the method comprising administering to a subject in need thereof a therapeutically effective dose of a multifunctional fusion of an IL-2 polypeptide, an IL-33 polypeptide, and a half-life extension moiety, wherein the half-life extension moiety is located at the N-terminus or C-terminus of the fusion protein. In some embodiments, the administration is subcutaneous, intramuscular or intravenous. In some embodiments, the administration is subcutaneous, intramuscular or intravenous. In some embodiments, the administration is subcutaneous. In some embodiments, the administration is intramuscular. In some embodiments, the administration is intravenous. Other routes of administration are discussed in greater detail in another section.

In some embodiments, the administration is subcutaneous. In some embodiments, the administration stimulates proliferation and/or activation of immune cells.

In some embodiments, the administration stimulates proliferation and/or activation of immune cells. In some embodiments, the immune cells are ST2+ cells, Foxp3-ST2-Tr1 cells, ILC2, macrophages, MDSCs, regulatory B cells, B1B cells, or tolerogenic dendritic cells.

In some embodiments, the administration promotes recruitment of pro-reparative immune cells. In some embodiments, the pro-reparative immune cells are Tregs, Th2 cells, M2 macrophages, MDSC, basophils, eosinophils, mast cells, or combinations thereof.

In some embodiments, the administration promotes immune activation for anti-cancer therapy. In some embodiments, the administration promotes activation of Th1, Th2, Th17, cytotoxic CD8 T cells, B, NK, NKT, gamma-delta T cells, ILC1, ILC2, ILC3, macrophages, dendritic cells, neutrophils, basophils, eosinophils, mast cells, or combinations thereof.

In some embodiments, the administration promotes immune activation against infectious diseases. In some embodiments, the administration promotes immune activation against infectious diseases via activation of Th1, Th2, Th17, cytotoxic CD8 T cells, B, NK, NKT, gamma-delta T cells, ILC1, ILC2, ILC3, macrophages, dendritic cells, neutrophils, basophils, eosinophils, mast cells, or combinations thereof.

In some embodiments, the administration promotes immune activation as a vaccine adjuvant for peptide, subunit, conjugate, mRNA, anti-cancer vaccines.

In some embodiments, provided herein is a method for detection or a diagnostic agent for soluble and membrane bound ST2.

### Routes of Administration

A multifunctional protein described herein (or a composition or medicament containing a recombinant multifunctional protein described herein) can be administered by any appropriate route. In some embodiments, a recombinant IL-33 variant or recombinant IL-2 and IL-33 variant multifunctional fusion protein or a pharmaceutical composition containing the same is administered systemically. Systemic administration may be intravenous, intradermal, inhalation, transdermal (topical), intraocular, intramuscular, subcutaneous, intramuscular, oral and/or transmucosal administration. In some embodiments, a recombinant IL-33 variant or recombinant IL-2 and IL-33 variant multifunctional fusion protein or a pharmaceutical composition containing the same is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, the thigh region, abdominal region, gluteal region, or scapular region. In some embodiments, a recombinant IL-33 variant or recombinant IL-2 and IL-33 variant multifunctional fusion protein or a pharmaceutical composition containing the same is administered intravenously. In some embodiments, a recombinant IL-33 variant or recombinant IL-2 and IL-33 variant multifunctional fusion protein or a pharmaceutical composition containing the same is administered orally. In some embodiments, a recombinant IL-33 variant or recombinant IL-2 and IL-33 variant multifunctional fusion protein or a pharmaceutical composition containing the same is administered intramuscularly. In some embodiments, more than one route can be used concurrently.

In some embodiments, administration results only in a localized effect in an individual, while in other embodiments, administration results in effects throughout multiple portions of an individual, for example, systemic effects. Typically, administration results in delivery of a recombinant IL-33 variant or multifunctional fusion protein systemically. In some embodiments, the recombinant IL-33 variant or multifunctional fusion protein is delivered to one or more target tissues including, but not limited to, heart, brain, spinal cord, striated muscle (e.g., skeletal muscle), smooth muscle, kidney, liver, lung, and/or spleen.

Combination therapies: In further embodiments, IL-2 and IL-33 variants or fusion proteins comprising the same described herein are administered in combination with other agents useful for treating a condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but also include treatment regimens in which a T-reg-selective IL-2 and/or IL-33 variant is administered at least once during a course of treatment that involves administering at least one other therapeutic agent to the patient. In certain embodiments, an IL-2 or IL-33 variant is administered in combination with an inhibitor of the PI3-K/AKT/mTOR pathway, e.g., rapamycin (Rapamune, sirolimus). Inhibitors of this pathway in combination with IL-2 and/or IL-33 favor T-reg enrichment.

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted being prior art to the claimed invention. In addition, the materials, methods, and examples are illustrative only and are not intended to be limiting.

TABLE 2
Sequences of IL-33 variants

| | | |
|---|---|---|
| SEQ ID NO: 1 | truncated wild | SITGISPITEYLASLSTYNDQSITFALEDESYEIYVEDLKKDE |
| | type human IL- | KKDKVLLSYYESQHPSNESGDGVDGKMLMVTLSPTKDFWLHAN |
| | 33 | NKEHSVELHKCEKPLPDQAFFVLHNMHSNCVSFECKTDPGVFI |
| | | GVKDNHLALIKVDSSENLCTENILFKLSET |
| SEQ ID NO: 2 | truncated N60S | SITGISPITEYLASLSTYNDQSITFALEDESYEIYVEDLKKDE |
| | human IL-33 | KKDKVLLSYYESQHPSSESGDGVDGKMLMVTLSPTKDFWLHAN |
| | | NKEHSVELHKCEKPLPDQAFFVLHNMHSNCVSFECKTDPGVFI |
| | | GVKDNHLALIKVDSSENLCTENILFKLSET |
| SEQ ID NO: 3 | truncated | SITGISPITEYLASLSTYNDQSITFALEDESYEIYVEDLKKDE |
| | C116F human | KKDKVLLSYYESQHPSNESGDGVDGKMLMVTLSPTKDFWLHAN |
| | IL-33 | NKEHSVELHKCEKPLPDQAFFVLHNMHSNFVSFECKTDPGVFI |
| | | GVKDNHLALIKVDSSENLCTENILFKLSET |
| SEQ ID NO: 4 | truncated C97G | SITGISPITEYLASLSTYNDQSITFALEDESYEIYVEDLKKDE |
| | human IL-33 | KKDKVLLSYYESQHPSNESGDGVDGKMLMVTLSPTKDFWLHAN |
| | | NKEHSVELHKGEKPLPDQAFFVLHNMHSNCVSFECKTDPGVFI |
| | | GVKDNHLALIKVDSSENLCTENILFKLSET |
| SEQ ID NO: 5 | truncated | SITGISPITEYLASLSTYNDQSITFALEDESYEIYVEDLKKDE |
| | N60S + C116F | KKDKVLLSYYESQHPSSESGDGVDGKMLMVTLSPTKDFWLHAN |
| | human IL-33 | NKEHSVELHKCEKPLPDQAFFVLHNMHSNFVSFECKTDPGVFI |
| | | GVKDNHLALIKVDSSENLCTENILFKLSET |
| SEQ ID NO: 6 | truncated | SITGISPITEYLASLSTYNDQSITFALEDESYEIYVEDLKKDE |
| | N60S + C97G | KKDKVLLSYYESQHPSSESGDGVDGKMLMVTLSPTKDFWLHAN |
| | human IL-33 | NKEHSVELHKGEKPLPDQAFFVLHNMHSNCVSFECKTDPGVFI |
| | | GVKDNHLALIKVDSSENLCTENILFKLSET |
| SEQ ID NO: 7 | truncated | SITGISPITEYLASLSTYNDQSITFALEDESYEIYVEDLKKDE |
| | C116F + C97G | KKDKVLLSYYESQHPSNESGDGVDGKMLMVTLSPTKDFWLHAN |
| | human IL-33 | NKEHSVELHKGEKPLPDQAFFVLHNMHSNFVSFECKTDPGVFI |
| | | GVKDNHLALIKVDSSENLCTENILFKLSET |
| SEQ ID NO: 8 | truncated | SITGISPITEYLASLSTYNDQSITFALEDESYEIYVEDLKKDE |
| | N60S + C116F + | KKDKVLLSYYESQHPSSESGDGVDGKMLMVTLSPTKDFWLHAN |
| | C97G human | NKEHSVELHKGEKPLPDQAFFVLHNMHSNFVSFECKTDPGVFI |
| | IL-33 | GVKDNHLALIKVDSSENLCTENILFKLSET |
| SEQ ID NO: 82 | truncated | SITGISPITEYLASLSTYNDQSITFALEDESYEIYVEDLKKDE |
| | N60S + C116F + | KKDKVLLSYYESQHPSSESGDGVDGKMLMVTLSPTKDFWLHAN |
| | C97G + C148G | NKEHSVELHKGEKPLPDQAFFVLHNMHSNFVSFECKTDPGVFI |
| | human IL-33 | GVKDNHLALIKVDSSENLGTENILFKLSET |
| SEQ ID NO: 83 | truncated | SITGISPITEYLASLSTYNDQSITFALEDESYEIYVEDLKKDE |
| | N60S + C116F + | KKDKVLLSYYESQHPSSESGDGVDGKMLMVTLSPTKDFWLHAN |
| | C97G + C121S + | NKEHSVELHKGEKPLPDQAFFVLHNMHSNFVSFESKTDPGVFI |
| | C148G human | GVKDNHLALIKVDSSENLGTENILFKLSET |
| | IL-33 | |

TABLE 3
Sequences of IL-2 variants

| | | |
|---|---|---|
| SEQ ID NO: 9 | human IL-2 | MYRMQLLSCIALSLALVTNSAPTSSSTKKTQLQLEHLLLDLQ |
| | with signal | MILNGINNYKNPKLTRMLTFKFYMPKKATELKHLQCLEEELK |
| | peptide | PLEEVLNLAQSKNFHLRPRDLISNINVIVLELKGSETTEMCE |
| | | YADETATIVEFLNRWITFCQSIISTLT |
| SEQ ID NO: 10 | wild type human | APTSSSTKKTQLQLEHLLLDLQMILNGINNYKNPKLTRMLTF |
| | IL-2 (mature) | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLT |
| SEQ ID NO: 11 | human IL-2 | APTSSSTKKTQLQLEHLLLDLQMILNGINNYKNPKLTRMLTF |
| | V69A | KFYMPKKATELKHLQCLEEELKPLEEALNLAQSKNFHLRPRD |
| | | LISNINVIVLELKGSETTFMCEYADETATIVEFLNRWITFCQ |
| | | SIISTLT |
| SEQ ID NO: 12 | human IL-2 | APTSSSTKKTQLQLEHLLLDLQMILNGINNYKNPKLTRMLTF |
| | Q74P | KFYMPKKATELKHLQCLEEELKPLEEVLNLAQSKNFHLRPRD |
| | | LISNINVIVLELKGSETTFMCEYADETATIVEFLNRWITFCQ |
| | | SIISTLT |
| SEQ ID NO: 13 | human IL-2 | APTSSSTKKTQLQLEHLLLDLQMILNGINNYKNPKLTRMLTF |
| | N88D | KFYMPKKATELKHLQCLEEELKPLEEVLNLAQSKNFHLRPRD |
| | | LISDINVIVLELKGSETTFMCEYADETATIVEFLNRWITFCQ |
| | | SIISTLT |
| SEQ ID NO: 14 | human IL-2 | APTSSSTKKTQLQLEHLLLDLQMILNGINNYKNPKLTRMLTF |
| | N88R | KFYMPKKATELKHLQCLEEELKPLEEVLNLAQSKNFHLRPRD |
| | | LISRINVIVLELKGSETTFMCEYADETATIVEFLNRWITFCQ |
| | | SIISTLT |
| SEQ ID NO: 15 | human IL-2 | APTSSSTKKTQLQLEHLLLDLQMILNGINNYKNPKLTRMLTF |
| | N88S | KFYMPKKATELKHLQCLEEELKPLEEVLNLAQSKNFHLRPRD |
| | | LISSINVIVLELKGSETTFMCEYADETATIVEFLNRWITFCQ |
| | | SIISTLT |
| SEQ ID NO: 16 | human IL-2 | APTSSSTKKTQLQLEHLLLDLQMILNGINNYKNPKLTRMLTF |
| | C125S | KFYMPKKATELKHLQCLEEELKPLEEVLNLAQSKNFHLRPRD |
| | | LISNINVIVLELKGSETTFMCEYADETATIVEFLNRWITFSQ |
| | | SIISTLT |
| SEQ ID NO: 17 | human IL-2 | APTSSSTKKTQLQLEHLLLDLQMILNGINNYKNPKLTRMLTF |
| | N88D + C125S | KFYMPKKATELKHLQCLEEELKPLEEVLNLAQSKNFHLRPRD |
| | | LISDINVIVLELKGSETTFMCEYADETATIVEFLNRWITFSQ |
| | | SIISTLT |
| SEQ ID NO: 18 | human IL-2 | APTSSSTKKTQLQLEHLLLDLQMILNGINNYKNPKLTRMLTF |
| | N88R + C125S | KFYMPKKATELKHLQCLEEELKPLEEVLNLAQSKNFHLRPRD |
| | | LISRINVIVLELKGSETTFMCEYADETATIVEFLNRWITFSQ |
| | | SIISTLT |
| SEQ ID NO: 19 | human IL-2 | APTSSSTKKTQLQLEHLLLDLQMILNGINNYKNPKLTRMLTF |
| | N88S + C125S | KFYMPKKATELKHLQCLEEELKPLEEVLNLAQSKNFHLRPRD |
| | | LISSINVIVLELKGSETTFMCEYADETATIVEFLNRWITFSQ |
| | | SIISTLT |
| SEQ ID NO: 20 | human IL-2 | APTSSSTKKTQLQLEHLLLDLQMILNGINNYKNPKLTRMLTF |
| | V69A + N88D + | KFYMPKKATELKHLQCLEEELKPLEEALNLAQSKNFHLRPRD |
| | C125S | LISDINVIVLELKGSETTFMCEYADETATIVEFLNRWITFSQ |
| | | SIISTLT |
| SEQ ID NO: 21 | human IL-2 | APTSSSTKKTQLQLEHLLLDLQMILNGINNYKNPKLTRMLTF |
| | V69A + N88R + | KFYMPKKATELKHLQCLEEELKPLEEALNLAQSKNFHLRPRD |
| | C125S | LISRINVIVLELKGSETTFMCEYADETATIVEFLNRWITFSQ |
| | | SIISTLT |
| SEQ ID NO: 22 | human IL-2 | APTSSSTKKTQLQLEHLLLDLQMILNGINNYKNPKLTRMLTF |
| | V69A + N88S + | KFYMPKKATELKHLQCLEEELKPLEEALNLAQSKNFHLRPRD |
| | C125S | LISSINVIVLELKGSETTFMCEYADETATIVEFLNRWITFSQ |
| | | SIISTLT |
| SEQ ID NO: 23 | human IL-2 | APTSSSTKKTQLQLEHLLLDLQMILNGINNYKNPKLTRMLTF |
| | Q74P + N88D + | KFYMPKKATELKHLQCLEEELKPLEEVLNLAPSKNFHLRPRD |
| | C125S | LISDINVIVLELKGSETTFMCEYADETATIVEFLNRWITFSQ |
| | | SIISTLT |
| SEQ ID NO: 24 | human IL-2 | APTSSSTKKTQLQLEHLLLDLQMILNGINNYKNPKLTRMLTF |
| | Q74P + N88D + | KFYMPKKATELKHLQCLEEELKPLEEVLNLAPSKNFHLRPRD |
| | C125S | LISDINVIVLELKGSETTFMCEYADETATIVEFLNRWITFSQ |
| | | SIISTLT |
| SEQ ID NO: 25 | human IL-2 | APTSSSTKKTQLQLEHLLLDLQMILNGINNYKNPKLTRMLTF |
| | Q74P + N88D + | KFYMPKKATELKHLQCLEEELKPLEEVLNLAPSKNFHLRPRD |
| | C125S | LISDINVIVLELKGSETTFMCEYADETATIVEFLNRWITFSQ |
| | | SIISTLT |
| SEQ ID NO: 26 | human IL-2 | APTSSSTKKTQLQLEHLLLDLQMILNGINNYKNPKLTRMLTF |
| | V69A + Q74P + | KFYMPKKATELKHLQCLEEELKPLEEALNLAPSKNFHLRPRD |
| | N88D + C125S | LISDINVIVLELKGSETTFMCEYADETATIVEFLNRWITFSQ |
| | | SIISTLT |
| SEQ ID NO: 27 | human IL-2 | APTSSSTKKTQLQLEHLLLDLQMILNGINNYKNPKLTRMLTF |
| | V69A + Q74P + | KFYMPKKATELKHLQCLEEELKPLEEALNLAPSKNFHLRPRD |
| | N88R + C125S | LISRINVIVLELKGSETTFMCEYADETATIVEFLNRWITFSQ |
| | | SIISTLT |
| SEQ ID NO: 28 | human IL-2 | APTSSSTKKTQLQLEHLLLDLQMILNGINNYKNPKLTRMLTF |
| | V69A + Q74P + | KFYMPKKATELKHLQCLEEELKPLEEALNLAPSKNFHLRPRD |
| | N88S + C125S | LISSINVIVLELKGSETTFMCEYADETATIVEFLNRWITFSQ |
| | | SIISTLT |

TABLE 4
Sequences of Linkers

| | | |
|---|---|---|
| SEQ ID NO: 29 | linker sequence | GGGGS |
| SEQ ID NO: 30 | linker sequence | GGGGSGGGGSGGGGS |
| SEQ ID NO: 31 | linker sequence | GGSGGGS |
| SEQ ID NO: 32 | linker sequence | EAAAK |
| SEQ ID NO: 33 | linker sequence | GGGGGGGG |
| SEQ ID NO: 34 | linker sequence | GSGSGSGSGS |
| SEQ ID NO: 35 | linker sequence | GSGSGSGSGSGSGSGSGSGS |
| SEQ ID NO: 36 | linker sequence | GGSGGSGGS |
| SEQ ID NO: 37 | linker sequence | GSGGS |
| SEQ ID NO: 38 | linker sequence | GSSGS |
| SEQ ID NO: 39 | linker sequence | GSAT |
| SEQ ID NO: 40 | linker sequence | SSSSGSSSSG |
| SEQ ID NO: 41 | linker sequence | LEGSGQGPGSGQGSGSPGSGQG |
| SEQ ID NO: 42 | linker sequence | GGGGSEAAAK |
| SEQ ID NO: 43 | linker sequence | GGGGSEAAAKGGGGSEAAAK |
| SEQ ID NO: 44 | linker sequence | EGKSSGSGSESKST |
| SEQ ID NO: 45 | linker sequence | GGGGSLVPRGSGGGGS |
| SEQ ID NO: 46 | linker sequence | KESGSVSSEQLAQFRSLD |
| SEQ ID NO: 47 | linker sequence | GGGSEGGGSEGGGSEGGG |
| SEQ ID NO: 48 | linker sequence | PAPAP |
| SEQ ID NO: 49 | linker sequence | AEAAAKEAAAKA |
| SEQ ID NO: 50 | linker sequence | AEAAAKEAAAKEAAAKEAAAKALEAEAAAKEAAAKEAAAK |
| | | EAAAKA |
| SEQ ID NO: 51 | CTP peptide | FQSSSS* KAPPPS*LPSPS*RLPGPS*DTPILPQ |
| SEQ ID NO: 52 | signal peptide | MGWSCIILFLVATATGVHS |
| SEQ ID NO: 53 | Flag tag | DYKDDDDK |
| SEQ ID NO: 54 | Wild-type Fc | DKTHTCPPCPAPELLGGPSVFLFPPKPKDTLMISRTPEVT |
| | (A77 or K77) | CVVVDVSHEDPEVKFNWYVDGVEVHNAKTKPREEQY[A/K] |
| | | STYRVVSVLTVLHQDWLNGKEYKCKVSNKALPAPIEKTI |
| | | SKAKGQPREPQVYTLPPSRDELTKNQVSLTCLVKGFYPSD |
| | | IAVEWESNGQPENNYKTTPPVLDSDGSFFLYSKLTVDKSR |
| | | WQQGNVFSCSVMHEALHNHYTQKSLSLSPGK |
| SEQ ID NO: 55 | Human albumin | DAHKSEVAHRFKDLGEENFKALVLIAFAQYLQQCPFEDHV |
| | binding domain | KLVNEVTEFAKTCVADESAENCDKSLHTLFGDKLCTVATL |
| | | RETYGEMADCCAKQEPERNECFLQHKDDNPNLPRLVRPEV |
| | | DVMCTAFHDNEETFLKKYLYEIARRHPYFYAPELLFFAKR |
| | | YKAAFTECCQAADKAACLLPKLDELRDEGKASSAKQRLKC |
| | | ASLQKFGERAFKAWAVARLSQRFPKAEFAEVSKLVTDLTK |
| | | VHTECCHGDLLECADDRADLAKYICENQDSISSKLKECCE |
| | | KPLLEKSHCIAEVENDEMPADLPSLAADFVESKDVCKNYA |
| | | EAKDVFLGMFLYEYARRHPDYSVVLLLRLAKTYETTLEKC |
| | | CAAADPHECYAKVFDEFKPLVEEPQNLIKQNCELFEQLGE |
| | | YKFQNALLVRYTKKVPQVSTPTLVEVSRNLGKVGSKCCKH |
| | | PEAKRMPCAEDYLSVVLNQLCVLHEKTPVSDRVTKCCTES |
| | | LVNRRPCFSALEVDETYVPKEFNAETFTFHADICTLSEKE |
| | | RQIKKQTALVELVKHKPKATKEQLKAVMDDFAAFVEKCCK |
| | | ADDKETCFAEEGKKLVAASQAALGL |
| SEQ ID NO: 56 | Human albumin | DAHKSEVAHRFKDLGEENFKALVLIAFAQYLQQCPFEDHV |
| | binding domain | KLVNEVTEFAKTCVADESAENCDKSLHTLFGDKLCTVATL |
| | QMP | RETYGEMADCCAKQEPERNECFLQHKDDNPNLPRLVRPEV |
| | | DVMCTAFHDNEETFLKKYLYEIARRHPYFYAPELLFFAKR |
| | | YKAAFTECCQAADKAACLLPKLDELRDEGKASSAKQRLKC |
| | | ASLQKFGERAFKAWAVARLSQRFPKAEFAEVSKLVTDLTK |
| | | VHTECCHGDLLECADDRADLAKYICENQDSISSKLKECCE |
| | | KPLLEKSHCIAEVENDEMPADLPSLAADFVESKDVCKNYA |
| | | EAKDVFLGMFLYEYARRHPDYSVVLLLRLAKTYETTLEKC |
| | | CAAADPHECYAKVFDEFKPLVEEPQNLIKQNCELFEQLGE |
| | | YKFQNALLVRYTKKVPQVSTPTLVEVSRNLGKVGSKCCKH |
| | | PEAKRMPCAEDYLSVVLNQLCVLHEKTPVSDRVTKCCTES |
| | | LVNRRPCFSALEVDETYVPKEFNAQTFTFHADICTLSEKE |
| | | RQIKKQMALVELVKHKPKATKEQLKAVMDDFAAFVEKCCK |
| | | ADDKETCFAEEGPKLVAASQAALGL |
| SEQ ID NO: 57 | Human albumin | MDFISIQQLVSGERVEGKVLGFGHGVPDPGAWPSDWRRGP |
| | domain DARP | QEAVAREKLKLEEEKKKKLERFNSTRFNLDNLADLENLVQ |
| | | RRKKRLRHRVPPRKPEPLVKPQSQAQVEPVGLEMFLKAAAE |
| | | NQEYLIDKYLTDGGDPNAHDKLHRTALHWACLKGHSQLVNK |
| | | LLVAGATVDARDLLDRTPVFWACRGGHLVILKQLLNQGARV |
| | | NARDKIGSTPLHVAVRTRHPDCLEHLIECGAHLNAQDKEGD |
| | | TALHEAVRHGSYKAMKLLLLYGAELGVRNAASVTPVQLARD |
| | | WQRGIREALQAHVAHPRTRC |

TABLE 5
Sequences of Multifunctional Fusion Proteins

| | | |
|---|---|---|
| SEQ ID NO: | IL233 Fusion | MGWSCIILFLVATATGVHSAPTSSSTKKTQLQLEHLLLDLQ |
| 58 | protein (SLT1) | MILNGINNYKNPKLTRMLTFKFYMPKKATELKHLQCLEEEL |
| | IL233 WT (signal | KPLEEVLNLAQSKNFHLRPRDLISNINVIVLELKGSETTFM |
| | peptide | CEYADETATIVEFLNRWITFCQSIISTLTGGGGSGGGGSGG |
| | underlined; WT | GGSSITGISPITEYLASLSTYNDQSITFALEDESYEIYVED |
| | IL-2 polypeptide | LKKDEKKDKVLLSYYESQHPSNESGDGVDGKMLMVTLSPTK |
| | in italics (N88); | DFWLHANNKEHSVELHKCEKPLPDQAFFVLHNMHSNCVSFE |
| | peptide linker in | CKTDPGVFIGVKDNHLALIKVDSSENLCTENILFKLSET |
| | bold italics; WT | |
| | IL-33 polypeptide | |
| | N60, C97, C116, | |
| | C121, C148) | |
| SEQ ID NO: | IL233 Fusion | MGWSCIILFLVATATGVHSAPTSSSTKKTQLQLEHLLLDLQ |
| 59 | protein (SLT2) | MILNGINNYKNPKLTRMLTFKFYMPKKATELKHLQCLEEEL |
| | N88D/WT N60, | KPLEEVLNLAQSKNFHLRPRDLISDINVIVLELKGSETTFM |
| | C97, C116, C121, | CEYADETATIVEFLNRWITFCQSIISTLTGGGGSGGGGSGG |
| | C148 | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNCVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 60 | protein (SLT3) | MILNGINNYKNPKLTRMLTFKFYMPKKATELKHLQCLEEEL |
| | N88D/N60S | KPLEEVLNLAQSKNFHLRPRDLISDINVIVLELKGSETTFM |
| | | CEYADETATIVEFLNRWITFCQSIISTLTGGGGSGGGGSGG |
| | | GGSSITGISPITEYLASLSTYNDQSITFALEDESYEIYVED |
| | | LKKDEKKDKVLLSYYESQHPSSESGDGVDGKMLMVTLSPTK |
| | | DFWLHANNKEHSVELHKCEKPLPDQAFFVLHNMHSNCVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 61 | protein (SLT4) | MILNGINNYKNPKLTRMLTFKFYMPKKATELKHLQCLEEEL |
| | N88D/C116F | KPLEEVLNLAQSKNFHLRPRDLISDINVIVLELKGSETTFM |
| | | CEYADETATIVEFLNRWITFCQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 62 | protein (SLT5) | MILNGINNYKNPKLTRMLTFKFYMPKKATELKHLQCLEEEL |
| | N88D/C97G | KPLEEVLNLAQSKNFHLRPRDLISDINVIVLELKGSETTFM |
| | | CEYADETATIVEFLNRWITFCQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKGEKPLPDQAFFVLHNMHSNCVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 63 | protein (SLT6) | MILNGINNYKNPKLTRMLTFKFYMPKKATELKHLQCLEEEL |
| | N88D/N60S + | KPLEEVLNLAQSKNFHLRPRDLISDINVIVLELKGSETTFM |
| | C116F + C97G | CEYADETATIVEFLNRWITFCQSIISTLTGGGGSGGGGSGG |
| | | GGSSITGISPITEYLASLSTYNDQSITFALEDESYEIYVED |
| | | LKKDEKKDKVLLSYYESQHPSSESGDGVDGKMLMVTLSPTK |
| | | DFWLHANNKEHSVELHKGEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 64 | protein (SLT7) | MILNGINNYKNPKLTRMLTFKFYMPKKATELKHLQCLEEEL |
| | N88D/C116F + | KPLEEVLNLAQSKNFHLRPRDLISDINVIVLELKGSETTFM |
| | C97G | CEYADETATIVEFLNRWITFCQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKGEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 65 | protein (SLT8) | MILNGINNYKNPKLTRMLTFKFYMPKKATELKHLQCLEEEL |
| | N88D + C125S/ | KPLEEVLNLAQSKNFHLRPRDLISDINVIVLELKGSETTFM |
| | C116F + C97G | CEYADETATIVEFLNRWITFSQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKGEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 66 | protein (SLT9) | MILNGINNYKNPKLTRMLTFKFYMPKKATELKHLQCLEEEL |
| | N88D + C125S/ | KPLEEVLNLAQSKNFHLRPRDLISDINVIVLELKGSETTFM |
| | C116F | CEYADETATIVEFLNRWITFSQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 67 | protein (SLT10) | MILNGINNYKNPKLTRMLTFKFYMPKKATELKHLQCLEEEL |
| | N88D + C125S/ | KPLEEVLNLAQSKNFHLRPRDLISDINVIVLELKGSETTFM |
| | C116F/Fc-K77A | CEYADETATIVEFLNRWITFSQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNFVSFE |
| | | CKTDPGVFIGVKDNHLALIKVDSSENLCTENILFKLSETGG |
| | | GGSGGGGSGGGGSDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKG |
| | | FYPSDIAVEWESNGQPENNYKTTPPVLDSDGSFFLYSKLTV |
| | | DKSRWQQGNVFSCSVMHEALHNHYTQKSLSLSPG |
| SEQ ID NO: | IL233 Fusion | MGWSCIILFLVATATGVHSAPTSSSTKKTQLQLEHLLLDLQ |
| 68 | protein (SLT11) | MILNGINNYKNPKLTRMLTFKFYMPKKATELKHLQCLEEEL |
| | N88 + C125/ | KPLEEVLNLAQSKNFHLRPRDLISNINVIVLELKGSETTFM |
| | C116F | CEYADETATIVEFLNRWITFCQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 69 | protein (SLT12) | MILNGINNYKNPKLTRMLTFKFYMPKKATELKHLQCLEEEL |
| | C125S/C116F/ | KPLEEVLNLAQSKNFHLRPRDLISNINVIVLELKGSETTFM |
| | Fc-K77A | CEYADETATIVEFLNRWITFSQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNFVSFE |
| | | CKTDPGVFIGVKDNHLALIKVDSSENLCTENILFKLSETGG |
| | | GGSGGGGSGGGGSDKTHTCPPCPAPELLGGPSVFLFPPKPK |
| | | DTLMISRTPEVTCVVVDVSHEDPEVKFNWYVDGVEVHNAKT |
| | | KPREEQYASTYRVVSVLTVLHQDWLNGKEYKCKVSNKALPA |
| | | PIEKTISKAKGQPREPQVYTLPPSRDELTKNQVSLTCLVKG |
| | | FYPSDIAVEWESNGQPENNYKTTPPVLDSDGSFFLYSKLTV |
| | | DKSRWQQGNVFSCSVMHEALHNHYTQKSLSLSPG |
| SEQ ID NO: | IL233 Fusion | MGWSCIILFLVATATGVHSDKTHTCPPCPAPELLGGPSVFL |
| 70 | protein (SLT13) | FPPKPKDTLMISRTPEVTCVVVDVSHEDPEVKFNWYVDGVE |
| | Fc-K77A, | VHNAKTKPREEQYASTYRVVSVLTVLHQDWLNGKEYKCKVS |
| | N88 + C125S/ | NKALPAPIEKTISKAKGQPREPQVYTLPPSRDELTKNQVSL |
| | C116F | TCLVKGFYPSDIAVEWESNGQPENNYKTTPPVLDSDGSFFL |
| | | YSKLTVDKSRWQQGNVFSCSVMHEALHNHYTQKSLSLSPGK |
| | | GGGGSGGGGSGGGGSAPTSSSTKKTQLQLEHLLLDLQMILN |
| | | GINNYKNPKLTRMLTFKFYMPKKATELKHLQCLEEELKPLE |
| | | EVLNLAQSKNFHLRPRDLISNINVIVLELKGSETTFMCEYA |
| | | DETATIVEFLNRWITFSQSIISTLTGGGGSGGGGSGGGGSS |
| | | ITGISPITEYLASLSTYNDQSITFALEDESYEIYVEDLKKD |
| | | EKKDKVLLSYYESQHPSNESGDGVDGKMLMVTLSPTKDFWL |
| | | HANNKEHSVELHKCEKPLPDQAFFVLHNMHSNFVSFECKTD |
| | | PGVFIGVKDNHLALIKVDSSENLCTENILFKLSET |
| SEQ ID NO: | IL233 Fusion | MGWSCIILFLVATATGVHSDKTHTCPPCPAPELLGGPSVFL |
| 71 | protein (SLT14) | FPPKPKDTLMISRTPEVTCVVVDVSHEDPEVKFNWYVDGVE |
| | Fc-K77/N88D + | VHNAKTKPREEQYASTYRVVSVLTVLHQDWLNGKEYKCKVS |
| | C125S/C116F | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| | | GGGGSGGGGSGGGGSAPTSSSTKKTQLQLEHLLLDLQMILN |
| | | GINNYKNPKLTRMLTFKFYMPKKATELKHLQCLEEELKPLE |
| | | EVLNLAQSKNFHLRPRDLISDINVIVLELKGSETTFMCEYA |
| | | DETATIVEFLNRWITFSQSIISTLTGGGGSGGGGSGGGGSS |
| | | ITGISPITEYLASLSTYNDQSITFALEDESYEIYVEDLKKD |
| | | EKKDKVLLSYYESQHPSNESGDGVDGKMLMVTLSPTKDFWL |
| | | HANNKEHSVELHKGEKPLPDQAFFVLHNMHSNFVSFECKTD |
| | | PGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 72 | protein (SLT15) | MILNGINNYKNPKLTRMLTFKFYMPKKATELKHLQCLEEEL |
| | V69A, Q74P, | KPLEEALNLAPSKNFHLRPRDLISDINVIVLELKGSETTFM |
| | N88D + C125S/ | CEYADETATIVEFLNRWITFSQSIISTLTGGGGSGGGGSGG |
| | C116F | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 73 | protein (SLT16) | MILNGINNYKNPKLTRMLTFKFYMPKKATELKHLQCLEEEL |
| | V69A, Q74P, | KPLEEALNLAPSKNFHLRPRDLISDINVIVLELKGSETTFM |
| | N88D + C125S/ | CEYADETATIVEFLNRWITFSQSIISTLTGGGGSGGGGSGG |
| | C97G + C116F | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKGEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSAPTSSSTKKTQLQLEHLLLDLQ |
| 74 | protein (SLT17) | MILNGINNYKNPKLTRMLTFKFYMPKKATELKHLQCLEEEL |
| | V69A, Q74P, | KPLEEALNLAPSKNFHLRPRDLISDINVIVLELKGSETTFM |
| | N88D + C125/ | CEYADETATIVEFLNRWITFCQSIISTLTGGGGSGGGGSGG |
| | C97 + C116F | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKCEKPLPDQAFFVLHNMHSNFVSFE |
| | | CKTDPGVFIGVKDNHLALIKVDSSENLCTENILFKLSET |
| SEQ ID NO: | IL233 Fusion | MGWSCIILFLVATATGVHSDYKDDDDKAPTSSSTKKTQLQL |
| 75 | protein (SLT18: | EHLLLDLQMILNGINNYKNPKLTRMLTFKFYMPKKATELKH |
| | SLT8 flag) | LQCLEEELKPLEEVLNLAQSKNFHLRPRDLISDINVIVLEL |
| | N88D + C125S/ | KGSETTFMCEYADETATIVEFLNRWITFSQSIISTLTGGGG |
| | C116F + C97G | SGGGGSGGGGSSITGISPITEYLASLSTYNDQSITFALEDE |
| | Flag | SYEIYVEDLKKDEKKDKVLLSYYESQHPSNESGDGVDGKML |
| | | MVTLSPTKDFWLHANNKEHSVELHKGEKPLPDQAFFVLHNM |
| | | HSNFVSFECKTDPGVFIGVKDNHLALIKVDSSENLCTENIL |
| | | FKLSET |
| SEQ ID NO: | IL233 Fusion | MGWSCIILFLVATATGVHSDYKDDDDKAPTSSSTKKTQLQL |
| 76 | protein (SLT19: | EHLLLDLQMILNGINNYKNPKLTRMLTFKFYMPKKATELKH |
| | SLT9 flag) | LQCLEEELKPLEEVLNLAQSKNFHLRPRDLISDINVIVLEL |
| | N88D + C125S/ | KGSETTFMCEYADETATIVEFLNRWITFSQSIISTLTGGGG |
| | C116F flag | SGGGGSGGGGSSITGISPITEYLASLSTYNDQSITFALEDE |
| | | SYEIYVEDLKKDEKKDKVLLSYYESQHPSNESGDGVDGKML |
| | | MVTLSPTKDFWLHANNKEHSVELHKCEKPLPDQAFFVLHNM |
| | | HSNFVSFECKTDPGVFIGVKDNHLALIKVDSSENLCTENIL |
| | | FKLSET |
| SEQ ID NO: | IL233 Fusion | MGWSCIILFLVATATGVHSDYKDDDDKAPTSSSTKKTQLQL |
| 77 | protein (SLT20: | EHLLLDLQMILNGINNYKNPKLTRMLTFKFYMPKKATELKH |
| | SLT15 flag) | LQCLEEELKPLEEALNLAPSKNFHLRPRDLISDINVIVLEL |
| | V69A, Q74P, | KGSETTFMCEYADETATIVEFLNRWITFSQSIISTLTGGGG |
| | N88D + C125S/ | SGGGGSGGGGSSITGISPITEYLASLSTYNDQSITFALEDE |
| | C116F Flag | SYEIYVEDLKKDEKKDKVLLSYYESQHPSNESGDGVDGKML |
| | | MVTLSPTKDFWLHANNKEHSVELHKCEKPLPDQAFFVLHNM |
| | | HSNFVSFECKTDPGVFIGVKDNHLALIKVDSSENLCTENIL |
| | | FKLSET |
| SEQ ID NO: | IL233 Fusion | MGWSCIILFLVATATGVHSDYKDDDDKAPTSSSTKKTQLQL |
| 78 | protein (SLT21: | EHLLLDLQMILNGINNYKNPKLTRMLTFKFYMPKKATELKH |
| | SLT16 flag) | LQCLEEELKPLEEALNLAPSKNFHLRPRDLISDINVIVLEL |
| | V69A, Q74P, | KGSETTFMCEYADETATIVEFLNRWITFSQSIISTLTGGGG |
| | N88D + C125S/ | SGGGGSGGGGSSITGISPITEYLASLSTYNDQSITFALEDE |
| | C97G + C116F | SYEIYVEDLKKDEKKDKVLLSYYESQHPSNESGDGVDGKML |
| | Flag | MVTLSPTKDFWLHANNKEHSVELHKGEKPLPDQAFFVLHNM |
| | | HSNFVSFECKTDPGVFIGVKDNHLALIKVDSSENLCTENIL |
| | | FKLSET |
| SEQ ID NO: | IL233 Fusion | MGWSCIILFLVATATGVHSDYKDDDDKAPTSSSTKKTQLQL |
| 79 | protein (SLT22: | EHLLLDLQMILNGINNYKNPKLTRMLTFKFYMPKKATELKH |
| | SLT17 flag) | LQCLEEELKPLEEALNLAPSKNFHLRPRDLISDINVIVLEL |
| | V69A, Q74P, | KGSETTFMCEYADETATIVEFLNRWITFCQSIISTLTGGGG |
| | N88D + C125/ | SGGGGSGGGGSSITGISPITEYLASLSTYNDQSITFALEDE |
| | C97 + C116F | SYEIYVEDLKKDEKKDKVLLSYYESQHPSNESGDGVDGKML |
| | Flag | MVTLSPTKDFWLHANNKEHSVELHKCEKPLPDQAFFVLHNM |
| | | HSNFVSFECKTDPGVFIGVKDNHLALIKVDSSENLCTENIL |
| | | FKLSET |
| SEQ ID NO. | IL233 Fusion | MGWSCIILFLVATATGVHSAPTSSSTKKTQLQLEHLLLDLQ |
| 84 | protein (SLT27) | MILNGINNYKNPKLTRMLTFKFYMPKKATELKHLQCLEEEL |
| | | KPLEEVLNLAQSKNFHLRPRDLISDINVIVLELKGSETTFM |
| | | CEYADETATIVEFLNRWITFSQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKGEKPLPDQAFFVLHNMHSNFVSFE |
| | | CKTDPGVFIGVKDNHLALIKVDSSENLGTENILFKLSET |
| SEQ ID NO. | IL233 Fusion | MGWSCIILFLVATATGVHSAPTSSSTKKTQLQLEHLLLDLQ |
| 85 | protein (SLT28) | MILNGINNYKNPKLTRMLTFKFYMPKKATELKHLQCLEEEL |
| | | KPLEEVLNLAQSKNFHLRPRDLISDINVIVLELKGSETTFM |
| | | CEYADETATIVEFLNRWITFSQSIISTLTGGGGSGGGGSGG |
| | | GGSSITGISPITEYLASLSTYNDQSITFALEDESYEIYVED |
| | | LKKDEKKDKVLLSYYESQHPSNESGDGVDGKMLMVTLSPTK |
| | | DFWLHANNKEHSVELHKGEKPLPDQAFFVLHNMHSNFVSFE |
| | | SKTDPGVFIGVKDNHLALIKVDSSENLGTENILFKLSET |

Signal peptides are underlined.
Flag tag is in bold font.
IL-2 polypeptide is in italics
Linker is bold and italics.
IL-33 polypeptide is in regular font.

Amino acid mutations are bold and underlined (WT residues at the same position are in bold).

Half-life extension moiety (e.g., Fc sequences) are in bold.

TABLE 6
Exemplary IL-33 Variant and WT with Signal Peptide and Tag

| | | |
|---|---|---|
| SEQ ID NO: 80 | IL-33 WT (C97, | MGWSCIILFLVATATGVHSDYKDDDDKSITGISPITEYLASLS |
| | C116, C121, | TYNDQSITFALEDESYEIYVEDLKKDEKKDKVLLSYYESQHPS |
| | C148) with | NESGDGVDGKMLMVTLSPTKDFWLHANNKEHSVELHKCEKPLP |
| | signal peptide | DQAFFVLHNMHSNCVSFECKTDPGVFIGVKDNHLALIKVDSSE |
| | and Flag tag | NLCTENILFKLSET |
| SEQ ID NO: 81 | Exemplary IL- | MGWSCIILFLVATATGVHSDYKDDDDKSITGISPITEYLASLS |
| | 33 variant | TYNDQSITFALEDESYEIYVEDLKKDEKKDKVLLSYYESQHPS |
| | (C97G and | NESGDGVDGKMLMVTLSPTKDFWLHANNKEHSVELHKGEKPLP |
| | C116F, C121, | DQAFFVLHNMHSNFVSFECKTDPGVFIGVKDNHLALIKVDSSE |
| | C148) with | NLCTENILFKLSET |
| | signal peptide | |
| | and Flag tag | |
| SEQ ID NO: 86 | Exemplary IL- | MGWSCIILFLVATATGVHSDYKDDDDKSITGISPITEYLASLS |
| | 33 variant | TYNDQSITFALEDESYEIYVEDLKKDEKKDKVLLSYYESQHPS |
| | (C97G C116F, | NESGDGVDGKMLMVTLSPTKDFWLHANNKEHSVELHKGEKPLP |
| | C121, C148G) | DQAFFVLHNMHSNFVSFECKTDPGVFIGVKDNHLALIKVDSSE |
| | with signal | NLGTENILFKLSET |
| | peptide and | |
| | Flag tag | |
| SEQ ID NO: 87 | Exemplary IL- | MGWSCIILFLVATATGVHSDYKDDDDKSITGISPITEYLASLS |
| | 33 variant | TYNDQSITFALEDESYEIYVEDLKKDEKKDKVLLSYYESQHPS |
| | (C97G, C116F, | NESGDGVDGKMLMVTLSPTKDFWLHANNKEHSVELHKGEKPLP |
| | C121S and | DQAFFVLHNMHSNFVSFESKTDPGVFIGVKDNHLALIKVDSSE |
| | C148G) with | NLGTENILFKLSET |
| | signal peptide | |
| | and Flag tag | |

### EXAMPLES

### Example 1: Design and Construction of IL-33 and IL-2 Multifunctional Proteins

This example illustrates the design and construction of exemplary IL-33 and IL-2 multifunctional proteins.

Standard recombinant DNA techniques were used to manipulate DNA (Sambrook et al., Molecular cloning: A laboratory manual; Cold Spring Harbor Laboratory Press, Cold Spring Harbor, N.Y., 1989) and design constructs containing nucleotide sequences encoding IL-33 and IL-2 proteins. Sequencing of double stranded DNA was carried out to confirm the nucleotide sequence of the constructs.

Exemplary nucleotide residues encoding the truncated WT IL-33 polypeptide (SEQ ID NO: 1) were mutated to generate exemplary IL-33 variant constructs. Exemplary IL-33 variants include at least one amino acid substitution in relation to the wild-type IL-33 protein selected from the group consisting of N60, C97, C116, C121, or C148 (positions numbered relative to truncated IL-33), and wherein the substitution at any one of the cysteines C97, C116, C121, or C148 is not a serine. Exemplary IL-33 variants also include IL-33 variants comprising an amino acid substitution at N60 and any one of C97, C116, C121, or C148. Exemplary IL-33 variants also include variants comprising an amino acid substitution at C116F and an additional amino acid at any one of C97, C121, or C148.

Included herein are IL-33 variants comprising at least one amino acid substitution selected from N60S, N60D, C97G, and C116F. Exemplary IL-33 variants comprise amino acid substitutions of C97G and C116F. Exemplary IL-33 variants are listed in Table 2.

Exemplary nucleotide residues encoding the WT IL-2 protein (SEQ ID NO: 10) were mutated to generate exemplary IL-2 variant constructs. Exemplary IL-2 variants include at least one amino acid substitution in relation to the wild-type IL-2 protein. Exemplary IL-2 variants are listed in Table 3.

For example, the IL-2 variant comprises one or more amino acid substitutions selected from in relation to the wild-type IL-2 selected from a group consisting of T3N, T3A, L12G, L12K, L12Q, L12S, Q13G, E15A, E15G, E15S, H16A, H16D, H16E, H16G, H16K, H16M, H16N, H16R, H16S, H16T, H16V, H16Y, L19A, L19D, L19E, L19G, L19N, L19R, L19S, L19T, L19V, D20A, D20E, D20H, D201, D20Y, D20F, D20G, D20T, D20W, M23R, V69A, Q47P, R81A, R81G, R81S, R81T, D84A, D84E, D84G, D84K, D841, D84M, D84Q D84R, D84S, D84T, S87R, N88A, N88D, N88R, N88E, N88I, N88F, N88G, N88M, N88R, N88S, N88V, N88W, V91D, V91E, V91K, V91G, V91N, V91R, V91S, 192K, 192R, E95G, C125A, C125S, Q1261, Q126L, and Q126F.

Exemplary IL-2 variants of the present invention, for example, comprise one or more amino acid substitutions selected from V69A, Q47P, N88D, N88S and C125S.

Further, multifunctional fusion proteins were constructed, where the IL-2 polypeptide and IL-33 polypeptide were joined, for example, by a linker (e.g., a peptide linker).

### Example 2. Expression and Purification of Multifunctional Proteins Comprising IL-2 and IL-33 Variants

This example illustrates the expression and purification of exemplary multifunctional proteins comprising IL-2 and IL-33 variants.

Gene fragments encoding an IL233 multifunctional protein, including any exemplary variant as described herein, were generated by synthetic gene synthesis and/or PCR from suitable templates and subcloned into standard mammalian expression vector. Exponentially growing bacterial cells such as E. coli or Chinese hamster ovary (CHO) cells were cotransfected with expression vectors and IL233 polypeptides were isolated and purified.

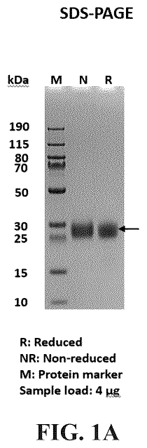

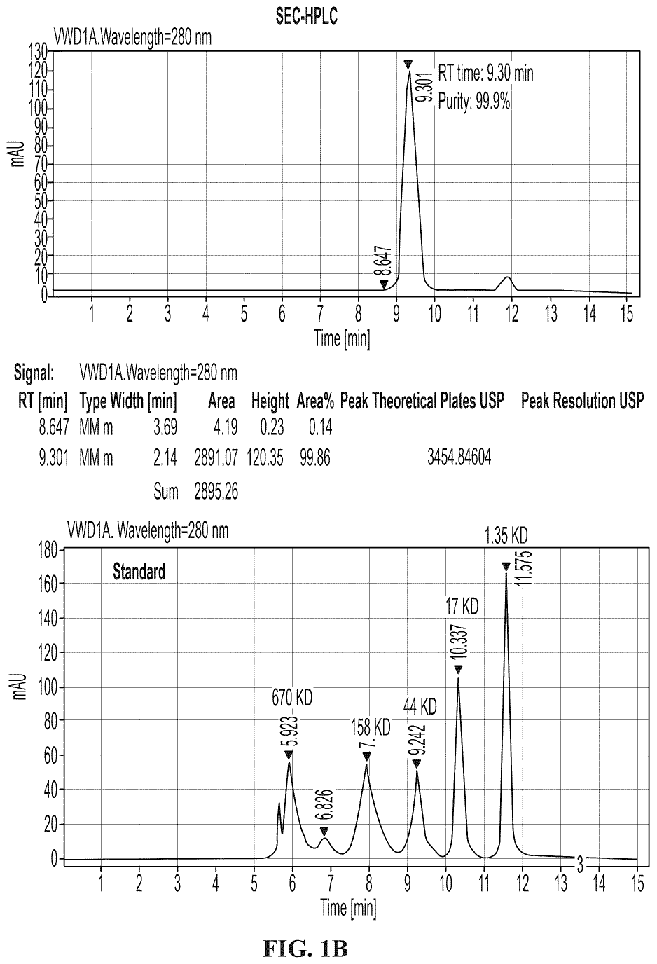

FIG. 1A is an SDS PAGE gel showing expression of an exemplary IL-33 variant, in reducing and non-reducing conditions.

FIG. 1B is a chromatogram showing a peak corresponding to 99.9% pure IL-33 variant.

Overall, the results showed good expression and purification of exemplary multifunctional protein comprising IL-2 and an IL-33 variant.

### Example 3. Activity and Potency of IL-33 Variants and IL-2 and IL-33 Multifunctional Proteins

This example illustrates the activity of exemplary IL-33 variant proteins, and exemplary IL-2 and IL-33 multifunctional proteins.

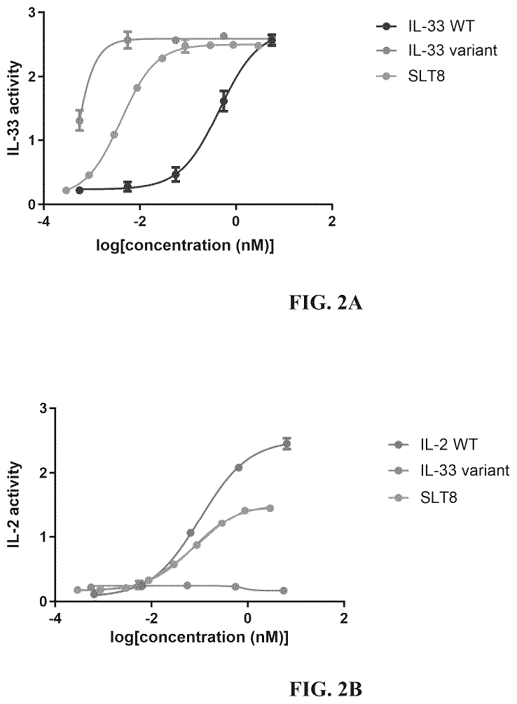

Briefly, IL-33 activity of an IL-33 variant, IL-33 WT and multifunctional protein comprising IL-2 and IL-33 were measured by NF-κB/AP-1-inducible secreted embryonic alkaline phosphatase (SEAP) reporter gene levels (indicative of IL-33 signaling) in an HEK-Blue™ IL-33 reporter cell line (InvivoGen). FIG. 2A is a graph showing robust IL-33 activity by an IL-33 variant polypeptide and an exemplary IL233 protein, SLT8 as compared to IL-33 WT.

Similarly, IL-2 activity of an IL-33 variant, IL-2 WT and multifunctional protein comprising IL-2 and IL-33 were measured by STAT5-inducible secreted embryonic alkaline phosphatase (SEAP) reporter gene levels (indicative of IL-2 signaling) in an HEK-Blue™ IL-33 reporter cell line (InvivoGen). FIG. 2B is a graph showing IL-2 activity of an exemplary IL233 protein, SLT8 relative to an IL-33 variant having no IL-2 activity and an IL-2 WT control showing robust activity.

The activity data was transformed and graphically plotted as a log of IL-33 concentration in ng/mL to calculate EC50 and measure potency.

TABLE 7
Relative Potency of IL-33 Variant, IL-33
WT and multifunctional fusion protein

| Sample | EC50 (nM) |
|---|---|
| IL-33 WT (SEQ ID NO: 81) | 0.4509 |
| IL-33 variant (SEQ ID NO: 80) | 0.0002999 |
| Fusion of IL-2 and IL-33 variant (SLT8) | 0.004017 |
| (SEQ ID NO: 65) | |

From the graph, the EC50 was determined as 0.0002999 for IL-33 variant, indicative of high potency. The multifunctional protein, SLT8 had an EC50 of 0.004017, also indicative of high potency. IL-33 WT had an EC50 of 0.4509. The results showed that an exemplary IL-33 variant of the present disclosure (SEQ ID NO: 80) had lower EC50 indicating higher potency in cells relative to truncated IL-33 WT (SEQ ID NO: 81). A multifunctional fusion protein (SEQ ID NO: 65) comprising an exemplary IL-33 variant also showed reduced EC50 relative to WT IL-33.

Overall, the results showed that IL-33 variants and multifunctional proteins comprising IL-33 variants and IL-2 had higher activity and potency in cells than truncated IL-33 WT.

### Example 4. Induction of ST2+ and Total Tregs by Exemplary Bifunctional Proteins, SLT8 and SLT27

Mice were subcutaneously injected with SLT8 or SLT27 (3 mice per study) at two different doses of 10 ug/kg or 100 ug/kg on day 0, 1 and 2. Pharmacodynamic analyses were carried out at day 4, 6 and 10. Complete blood count for measuring total number of eosinophils was carried out at day 4, 6, 10. A PBS control was obtained on day 4 after injection.

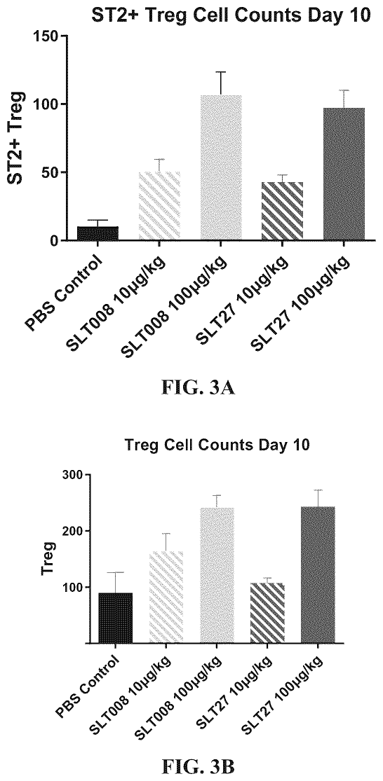

FIG. 3A is a bar graph showing the number of ST2+ Treg cells at day 10, measured from mice subcutaneously injected with a dose of 10 ug/kg or 100 ug/kg of exemplary bifunctional protein, SLT8 or SLT27 at days 0, 1 or 2. Both SLT8 and SLT27 showed a dose-dependent expansion of ST2+ Treg cells by about 5-10 fold. FIG. 3B is a bar graph showing the number of total Treg cells at day 10, measured from mice subcutaneously injected with a dose of 10 ug/kg or 100 ug/kg of exemplary bifunctional protein, SLT8 or SLT27 at days 0, 1 or 2. Both SLT8 and SLT27 showed a dose-dependent expansion of total Treg cells by about 2-3 fold.

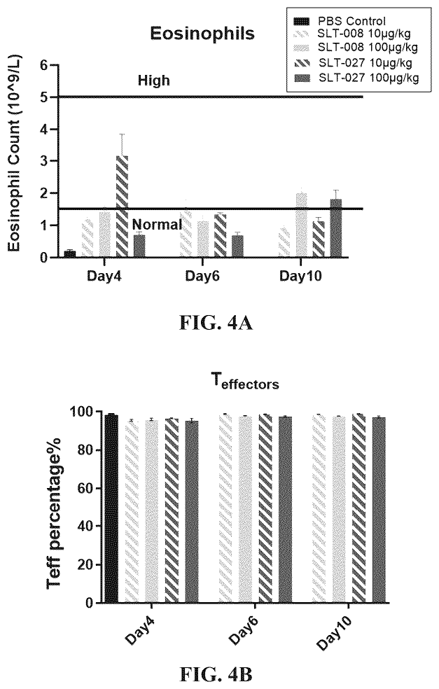

FIG. 4A is a bar graph of the number of eosinophils measured at days 4, 6 and 10 from mice subcutaneously injected with a dose of 10 μg/kg or 100 μg/kg of exemplary bifunctional protein, SLT8 or SLT27 at days 0, 1 or 2. SLT8 and SLT27 showed comparable amounts of eosinophils at the timepoints tested. A normal range for eosinophils is between 0-0.5×10\hat{ }9/L. Greater than 5×10\hat{ }9/L is considered high. Even with a moderate transient increase in eosinophils at day 4, total eosinophil levels remained low, indicative of low immunogenicity.

FIG. 4B is a bar graph of the percentage of T effector cells measured at days 4, 6 and 10 from mice subcutaneously injected with a dose of 10 ug/kg or 100 ug/kg of exemplary bifunctional protein, SLT8 or SLT27 at days 0, 1 or 2. SLT8 and SLT27 showed comparable amounts of T effector cells at the timepoints tested.

Overall, the results showed that SLT8 and SLT27 showed comparable expansion of ST2+ Treg and total Tregs across the timepoints and doses tested. The results also showed that selectivity of Treg to eosinophils or T_{effector} cells was comparable between SLT8 and SLT27. Eosinophil levels remained low, indicative of low immunogenicity.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A recombinant human interleukin-33 (IL-33) variant comprising one, two, three, four or five amino acid substitutions at N60, C97, C116, C121, and C148, wherein the IL-33 has improved activity and/or manufacturability relative to wild-type IL-33.

2. A recombinant human interleukin-33 (IL-33) variant comprising an amino acid substitution at N60 and any one of C97, C116, C121 or C148.

3. A recombinant human interleukin-33 (IL-33) variant comprising an amino acid substitution C116F and an additional amino acid at any one of C97, C121 or C148.

4. A recombinant human interleukin-33 (IL-33) variant comprising at least one amino acid substitution selected from N60S, N60D, N60T, N60A, N60Q, N60E C97G, C97A, C97V, C116A, C116F, C116Y, C121A, C121S, C148A, C148G, C148S, C148Y or C148N.

5. The recombinant human IL-33 variant of any one of the preceding claims comprising the amino acid substitutions of C97G and C116F.

6. The recombinant human IL-33 variant of claim 5, further comprising the amino acid substitutions of C148G.

7. The recombinant human IL-33 variant of claim 6 further comprising the amino acid substitutions of C121S.

8. The recombinant human IL-33 variant of any one of the preceding claims, wherein the IL-33 is truncated.

9. The recombinant human IL-33 variant of any one of the preceding claims, wherein the polypeptide comprises the sequence of any one of SEQ ID NOs: 2-8, and SEQ ID NOs: 82-83.

10. A fusion protein comprising IL-33 of any one of the preceding claims.

11. The fusion protein of claim 10, further comprising IL-2.

12. The fusion protein of any one of the preceding claims, further comprising a half-life extension moiety.

13. A multifunctional fusion protein comprising:
an IL-2 polypeptide,
an IL-33 polypeptide, and
a half-life extension moiety,
wherein the half-life extension moiety is located at the N-terminus or C-terminus of the fusion protein.

14. A multifunctional fusion protein comprising:
an IL-2 polypeptide,
an IL-33 polypeptide, and
a half-life extension moiety,
wherein the half-life extension moiety is not an Fc domain of IgG.

15. The multifunctional fusion protein of any one of the preceding claims, wherein the IL-33 polypeptide is a variant wherein the variant has improved activity and/or manufacturability relative to wild-type IL-33.

16. The multifunctional fusion protein of any one of the preceding claims, comprising one, two, three, four or five amino acid substitutions at N60, C97, C116, C121, and C148.

17. The multifunctional fusion protein of any one of claim 13 or 14, comprising an amino acid substitution at N60 and any one of C97, C116, C121, or C148.

18. The multifunctional fusion protein of any one of claim 13 or 14, comprising an amino acid substitution at C116F and an additional amino acid at any one of C97, C121, or C148.

19. The multifunctional protein of any one of claim 14 or 15, comprising at least one amino acid substitution selected from N60S, N60D, N60A, N60T, N60Q, N60E C97G, C97A, C97V, C116A, C116F, C116Y, C121A, C121S, C148A, C148G, C148S, C148Y or C148N.

20. The multifunctional protein of any one of the preceding claims, comprising the amino acid substitutions of C97G and C116F.

21. The multifunctional protein of claim 20, further comprising the amino acid substitution of C148G.

22. The multifunctional protein of claim 21, further comprising the amino acid substitution of C121S.

23. The multifunctional fusion protein of any one of the preceding claims, wherein the IL-33 is truncated.

24. The multifunctional fusion protein of any one of the preceding claims, wherein the IL-33 polypeptide comprises the sequence of any one of SEQ ID NO: 2-8, and SEQ ID NOs: 82-83.

25. The multifunctional fusion protein of any one of the preceding claims, wherein the IL-2 polypeptide is a variant IL-2.

26. The multifunctional fusion protein of any one of the preceding claims, wherein the IL-2 polypeptide comprises N88R, N88S, C125S, V69A or Q74P.

27. The multifunctional fusion protein of any one of the preceding claims, wherein the half-life extension moiety is selected from the group consisting of monomethoxy poly-ethylene glycol (PEG), albumin, transferrin, Fc domain of IgG, highly sialylated peptide, XTEN, V-P-G-x-G (ELP), proline-alanine-serine (PAS) polymer, homo amino acid polymer (HAP) and gelatin-like protein (GLK).

28. The PEG of claim 27, wherein the PEG is linear chain or branched chain.

29. The multifunctional fusion protein of claim 27, wherein the albumin is human serum albumin (HSA).

30. The multifunctional fusion protein of claim 27, wherein the highly sialylated peptide is carboxy terminal peptide (CTP) consisting of the sequence of FQSSSS* KAPPPS*LPSPS*RLPGPS*DTPILPQ (SEQ ID NO: 51).

31. The multifunctional fusion protein of claim 27, wherein the homo amino acid polymer consists of glycine.

32. The multifunctional fusion protein of claim 27, wherein the Fc domain comprises 90% identity to SEQ ID NO: 54.

33. The multifunctional fusion protein of claim 27, wherein the Fc domain comprises 100% identity to the wild-type sequence of SEQ ID NO: 54.

34. The multifunctional fusion protein of claim 27, wherein the Fc domain comprises one or more mutations that reduce effector function.

35. The multifunctional fusion protein of claim 27, wherein the one or more mutations are selected from N297A, N297Q, N297G, L234A, L235E, G237A, P238S, H268Q, H268A, V309L, A330S and P331S.

36. The multifunctional fusion protein of any one of the preceding claims, wherein the IL-2 and IL-33 polypeptides are 10 to 100 angstrom units (Å) apart.

37. The multifunctional fusion protein of any one of the preceding claims, wherein the multifunctional protein further comprises a linker.

38. The multifunctional fusion protein of claim 37, wherein the linker is a peptide linker or a chemical linker.

39. The multifunctional fusion protein of claim 37, wherein the linker comprises a sequence of GGGGS (SEQ ID NO: 29) or GGGGSGGGGSGGGGS (SEQ ID NO: 30).

40. The multifunctional fusion protein of any one of the preceding claims, further comprising a tag at the N-terminus or C-terminus.

41. The multifunctional fusion protein of claim 40, wherein the tag is a Flag tag.

42. The multifunctional fusion protein of any one of the preceding claims comprising the sequence of any one of SEQ ID NO: 58-79.

43. The multifunctional fusion protein of any one of the preceding claims, wherein the fusion protein is lyophilized.

44. A polynucleotide encoding the IL-33 polypeptide or the multifunctional fusion protein of any one of the preceding claims.

45. A vector comprising the polynucleotide of claim 44.

46. The vector of claim 45, wherein the vector is a viral vector or a non-viral vector.

47. The vector of claim 46, wherein the viral vector is an adeno-associated virus vector, an adenovirus vector or a lentivirus vector.

48. An engineered host cell comprising the polynucleotide of claim 44 or the vector of claims 45 to 47.

49. The engineered cell of claim 48, wherein the cell is a mammalian cell.

50. The engineered cell of claim 48, wherein the cell is a bacterial cell.

51. A microparticle comprising the polynucleotide of claim 44 or the multifunctional fusion protein of any one of claims 11-43.

52. The microparticle of claim 51, wherein the microparticle is a liposome, extracellular vesicle or a lipid nanoparticle.

53. A pharmaceutical composition comprising the multifunctional fusion protein of any one of the preceding claims.

54. A method of treating autoimmune disease, the method comprising administering to a subject in need thereof a therapeutically effective dose of the multifunctional fusion protein of any one of claims 11-43 or the pharmaceutical composition of claim 51 or the engineered cell of any one of claims 48-50.

55. A method of treating an autoimmune disease, the method comprising administering to a subject in need thereof a therapeutically effective dose of a multifunctional fusion of an IL-2 polypeptide, an IL-33 polypeptide, and a half-life extension moiety, wherein the half-life extension moiety is located at the N-terminus or C-terminus of the fusion protein.

56. The method of any one of claim 54 or 55, wherein the administration is subcutaneous, intramuscular or intravenous.

57. The method of claim 56, wherein the administration is subcutaneous.

58. The method of any one of the preceding claims, wherein the administration stimulates proliferation and/or activation of immune cells.

59. The method of any one of the preceding claims, wherein the immune cells are ST2+ cells, Foxp3-ST2+ and Foxp3-ST2-Tr1 cells, Th3 cells, ILC2, macrophages, MDSCs, regulatory B cells, B1B cells, or tolerogenic dendritic cells.

60. The method of claim 59, wherein the ST2+ cells are ST2⁺ T regulatory cells, tissue resident ST2⁺ cells or a combination of tissue resident ST2⁺ and CD25^{bright} cells.

61. The method of claim 60, wherein the proliferation and/or activation of ST2+ T regulatory cells is at least greater than 100-fold relative to a control.

62. The method of any one of the preceding claims, wherein the administration leads to coexpression of IL-2 and IL-33 in the same tissue.

63. The method of any one of the preceding claims, wherein IL-2 and IL-33 synergistically show increased potency and/or increased avidity relative to IL-2 or IL-33 alone.

64. The method of any one of the preceding claims, wherein administration increases local immune tolerance in a tissue microenvironment.


SEQUENCE LISTING (87 sequences)

SEQ ID NO: 1 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 2 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 3 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 4 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 5 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 6 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 7 - protein, 159 aa, synthetic construct
SITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 8 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 9 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, synthetic construct [sequence omitted]

SEQ ID NO: 11 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISNINVIVLELKGSETTFMCEYADETATIVEFLNRWITFCQSIISTLT

SEQ ID NO: 12 - protein, 133 aa, synthetic construct [sequence omitted]

SEQ ID NO: 13 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLT

SEQ ID NO: 14 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFCQSIISTLT

SEQ ID NO: 15 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 16 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 17 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 18 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 19 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISSINVIVLELKGSETTFMCEYADETATIVEFLNRWITFSQSIISTLT

SEQ ID NO: 20 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISDINVIVLELKGSETTFMCEYADETATIVEFLNRWITFSQSIISTLT

SEQ ID NO: 21 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISRINVIVLELKGSETTFMCEYADETATIVEFLNRWITFSQSIISTLT

SEQ ID NO: 22 - protein, 133 aa, synthetic construct
APTSSSTKKTQLQLEHLLLDLQMILNGINNYKNPKLTRMLTFKFYMPKKATELKHLQCLEEELKPLEEALNLAQSKNFHLRPRDLISSINVIVLELKGSETTFMCEYADETATIVEFLNRWITFSQSIISTLT

SEQ ID NO: 23 - protein, 133 aa, synthetic construct
APTSSSTKKTQLQLEHLLLDLQMILNGINNYKNPKLTRMLTFKFYMPKKATELKHLQCLEEELKPLEEVLNLAPSKNFHLRPRDLISDINVIVLELKGSETTFMCEYADETATIVEFLNRWITFSQSIISTLT

SEQ ID NO: 24 - protein, 133 aa, synthetic construct; identical to SEQ ID NO: 23

SEQ ID NO: 25 - protein, 133 aa, synthetic construct; identical to SEQ ID NO: 23

SEQ ID NO: 26 - protein, 133 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLT

SEQ ID NO: 27 - protein, 133 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISRINVIVLELKGSETTFMCEYADETATIVEFLNRWITFSQSIISTLT

SEQ ID NO: 28 - protein, 133 aa, synthetic construct
APTSSSTKKTQLQLEHLLLDLQMILNGINNYKNPKLTRMLTFKFYMPKKATELKHLQCLEEELKPLEEALNLAPSKNFHLRPRDLISSINVIVLELKGSETTFMCEYADETATIVEFLNRWITFSQSIISTLT

SEQ ID NO: 29 - protein, 5 aa, synthetic construct
GGGGS

SEQ ID NO: 30 - protein, 15 aa, synthetic construct
GGGGSGGGGSGGGGS

SEQ ID NO: 31 - protein, 7 aa, synthetic construct
GGSGGGS

SEQ ID NO: 32 - protein, 5 aa, synthetic construct
EAAAK

SEQ ID NO: 33 - protein, 8 aa, synthetic construct
GGGGGGGG

SEQ ID NO: 34 - protein, 10 aa, synthetic construct
GSGSGSGSGS

SEQ ID NO: 35 - protein, 20 aa, synthetic construct
GSGSGSGSGSGSGSGSGSGS

SEQ ID NO: 36 - protein, 9 aa, synthetic construct
GGSGGSGGS

SEQ ID NO: 37 - protein, 5 aa, synthetic construct
GSGGS

SEQ ID NO: 38 - protein, 5 aa, synthetic construct
GSSGS

SEQ ID NO: 39 - protein, 4 aa, synthetic construct
GSAT

SEQ ID NO: 40 - protein, 10 aa, synthetic construct
SSSSGSSSSG

SEQ ID NO: 41 - protein, 22 aa, synthetic construct
LEGSGQGPGSGQGSGSPGSGQG

SEQ ID NO: 42 - protein, 10 aa, synthetic construct
GGGGSEAAAK

SEQ ID NO: 43 - protein, 20 aa, synthetic construct
GGGGSEAAAKGGGGSEAAAK

SEQ ID NO: 44 - protein, 14 aa, synthetic construct
EGKSSGSGSESKST

SEQ ID NO: 45 - protein, 16 aa, synthetic construct
GGGGSLVPRGSGGGGS

SEQ ID NO: 46 - protein, 18 aa, synthetic construct
KESGSVSSEQLAQFRSLD

SEQ ID NO: 47 - protein, 18 aa, synthetic construct
GGGSEGGGSEGGGSEGGG

SEQ ID NO: 48 - protein, 5 aa, synthetic construct
PAPAP

SEQ ID NO: 49 - protein, 12 aa, synthetic construct
AEAAAKEAAAKA

SEQ ID NO: 50 - protein, 46 aa, synthetic construct
AEAAAKEAAAKEAAAKEAAAKALEAEAAAKEAAAKEAAAKEAAAKA

SEQ ID NO: 51 - protein, 30 aa, synthetic construct
FQSSSSKAPPPSLPSPSRLPGPSDTPILPQ

SEQ ID NO: 52 - protein, 19 aa, synthetic construct
MGWSCIILFLVATATGVHS

SEQ ID NO: 53 - protein, 8 aa, synthetic construct
DYKDDDDK

SEQ ID NO: 54 - protein, 227 aa, synthetic construct
  VARIANT 77: note = X may be A or K
DKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 585 aa, synthetic construct [sequence omitted]

SEQ ID NO: 56 - protein, 585 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAQTFTFHADICTLSEKERQIKKQMALVELVKHKPKATKEQLKAVMDDFAAFVEKCCKADDKETCFAEEGPKLVAASQAALGL

SEQ ID NO: 57 - protein, 305 aa, synthetic construct
MDFISIQQLVSGERVEGKVLGFGHGVPDPGAWPSDWRRGPQEAVAREKLKLEEEKKKKLERFNSTRFNLDNLADLENLVQRRKKRLRHRVPPRKPEPLVKPQSQAQVEPVGLEMFLKAAAENQEYLIDKYLTDGGDPNAHDKLHRTALHWACLKGHSQLVNKLLVAGATVDARDLLDRTPVFWACRGGHLVILKQLLNQGARVNARDKIGSTPLHVAVRTRHPDCLEHLIECGAHLNAQDKEGDTALHEAVRHGSYKAMKLLLLYGAELGVRNAASVTPVQLARDWQRGIREALQAHVAHPRTRC

SEQ ID NO: 58 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 59 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 60 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSSESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 61 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 62 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNCVSFECKTDPGVFIGVKDNHLALIKVDSSENLCTENILFKLSET

SEQ ID NO: 63 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 64 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 65 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 66 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 67 - protein, 567 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSETGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 69 - protein, 567 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSETGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 568 aa, synthetic construct
MGWSCIILFLVATATGVHSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 71 - protein, 568 aa, synthetic construct
MGWSCIILFLVATATGVHSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 72 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 73 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 74 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 75 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 76 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 77 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 78 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 79 - protein, 334 aa, synthetic construct
MGWSCIILFLVATATGVHSDYKDDDDKAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFCQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNFVSFECKTDPGVFIGVKDNHLALIKVDSSENLCTENILFKLSET

SEQ ID NO: 80 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 81 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLCTENILFKLSET

SEQ ID NO: 82 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFECKTDPGVFIGVKDNHLALIKVDSSENLGTENILFKLSET

SEQ ID NO: 83 - protein, 159 aa, synthetic construct
SITGISPITEYLASLSTYNDQSITFALEDESYEIYVEDLKKDEKKDKVLLSYYESQHPSSESGDGVDGKMLMVTLSPTKDFWLHANNKEHSVELHKGEKPLPDQAFFVLHNMHSNFVSFESKTDPGVFIGVKDNHLALIKVDSSENLGTENILFKLSET

SEQ ID NO: 84 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLGTENILFKLSET

SEQ ID NO: 85 - protein, 326 aa, synthetic construct
MGWSCIILFLVATATGVHSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFESKTDPGVFIGVKDNHLALIKVDSSENLGTENILFKLSET

SEQ ID NO: 86 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFECKTDPGVFIGVKDNHLALIKVDSSENLGTENILFKLSET

SEQ ID NO: 87 - protein, 186 aa, synthetic construct
MGWSCIILFLVATATGVHSDYKDDDDKSITGISPITEYLASLSTYNDQSITFALEDESYEIYVEDLKKDEKKDKVLLSYYESQHPSNESGDGVDGKMLMVTLSPTKDFWLHANNKEHSVELHKGEKPLPDQAFFVLHNMHSNFVSFESKTDPGVFIGVKDNHLALIKVDSSENLGTENILFKLSET